(19) United States
(12) Patent Application Publication
(10) Pub. No.: US 2024/0317609 A1
(43) Pub. Date: Sep. 26, 2024

(54) SLUDGE TREATMENT FACILITY OPERATION ASSISTANCE NAVIGATION SYSTEM AND SLUDGE TREATMENT FACILITY OEPRATION ASSISTANCE METHOD

(71) Applicant: Hitachi, Ltd., Tokyo (JP)

(72) Inventors: Jun TANAKA, Tokyo (JP); Yasuko KOBAYASHI, Tokyo (JP); Takuya KAMBAYASHI, Tokyo (JP); Daisuke YAGI, Tokyo (JP); Makiko UDAGAWA, Tokyo (JP); Kotaro KITAMURA, Tokyo (JP); Hiroki MIYAKAWA, Tokyo (JP)

(73) Assignee: Hitachi, Ltd., Tokyo (JP)

(21) Appl. No.: 18/269,713

(22) PCT Filed: Feb. 4, 2022
(86) PCT No.: PCT/JP2022/004567

§ 371 Date: Jun. 26, 2023

(30) Foreign Application Priority Data
Feb. 8, 2021 (JP) 2021-018635
Jun. 23, 2021 (JP) 2021-103846

(51) Int. Cl. C02F 1/00; C02F 11/147

(52) U.S. Cl. CPC C02F 1/008; C02F 11/147; C02F 2209/006; C02F 2209/008; C02F 2209/02; C02F 2209/44

(57) ABSTRACT

A sludge treatment facility operation assistance navigation system acquires an operating flock state that is a flocculation flock state during operation of a sludge treatment facility, acquires an operating sludge water content that is a water content of dehydrated sludge during operation of the sludge treatment facility, selects or generates facility operation contents of a flocculation tank and a sludge dehydrator based on the operating flock state, the operating sludge water content, a desired flocculation flock state, and a desired sludge water content, and displays the facility operation contents on a display device.

### TECHNICAL FIELD

The present invention relates to a navigation system and an operation assistance method for assisting an operation management work of a sludge flocculation facility and a dehydration facility in particular, in a sewage sludge treatment facility of a sewage treatment plant as an example.

### BACKGROUND ART

Regarding the dehydration of sludge, there has been proposed a sludge drying device including a pump for supplying sludge containing moisture to be removed, a centrifugal thin-film drying device for expanding the sludge into a thin film and removing the moisture contained therein by centrifugal force, a dried sludge collection container for collecting dried sludge dried by the centrifugal thin-film drying device, an infrared water content meter for irradiating the dried sludge with infrared rays to measure a water content, and a control device for controlling a rotation speed of the centrifugal thin-film drying device by an output of the infrared water content meter (PTL 1).

### CITATION LIST

### Patent Literature

PTL 1: JP H09-57296 A

### SUMMARY OF INVENTION

### Technical Problem

A sludge generation amount in a sewage treatment plant is enormous, a burden of sludge disposal is increased, and high efficiency of sludge treatment is required. In sewage treatment, generally, sludge concentration, concentrated sludge storage, sludge flocculation, sludge dehydrating, and dehydrated sludge storage and treatment steps are continued, and particularly, a sludge dehydrating step of reducing the water content of sludge by squeezing sludge to separate water and solids, and a flocculation step of flocculating suspended substances in sewage to form flocs as aggregate particles before the sludge dehydrating step to form a sludge form suitable for dehydrating, which are main treatment steps, are steps that have many problems in promoting high efficiency of sludge treatment.

Here, in order to obtain sludge having a water content reduced to a certain range after dehydration, it is important to manage dehydration and flocculation under a condition combining a flocculation step of adding a flocculant before the sludge dehydrating step to flocculate suspended substances in sewage to form flocs to be aggregate particles and the sludge dehydrating step described above. However, PTL 1 does not specifically disclose managing flocculation.

Therefore, an object of the present invention is to provide, as an example, in a sewage treatment facility in a sewage treatment plant, a sludge treatment facility operation assistance navigation system and a sludge treatment facility operation assistance method as operation assistance and control technique for a sludge treatment facility to improve the efficiency of the sludge treatment, the sludge treatment facility operation assistance navigation system and the sludge treatment facility operation assistance method being capable of displaying information for performing appropriate operation management with respect to both facilities for the main sludge dehydrating step and flocculation step related to the sludge treatment, and performing appropriate facility operation even for workers with poor experience values.

### Solution to Problem

According to a first aspect of the present invention for achieving the above object, there is provided the following sludge treatment facility operation assistance navigation system. That is, the sludge treatment facility operation assistance navigation system includes a processor, a storage resource, and a display device. The storage resource stores, as data, a desired flocculation flock state that is a desired flocculation flock state in a flocculation tank of a sludge treatment facility, and a desired sludge water content that is a desired water content of dehydrated sludge discharged from a sludge dehydrator of the sludge treatment facility. The processor executes an operation content display program to (1) acquire an operating flock state that is a flocculation flock state during operation of the sludge treatment facility. (2) An operating sludge water content, which is a water content of dehydrated sludge during operation of the sludge treatment facility, is acquired. (3) Facility operation contents of the flocculation tank and the sludge dehydrator is selected or generated based on the operating flock state, the operating sludge water content, the desired flocculation flock state, and the desired sludge water content. (4) The facility operation contents are displayed on the display device.

According to a second aspect of the present invention for achieving the above object, there is provided the following sludge treatment facility operation assistance method. That is, the sludge treatment facility operation assistance method uses an electronic computer. This sludge treatment facility operation assistance method (1) obtains an operating flock state that is a flocculation flock state during operation of a sludge treatment facility. (2) An operating sludge water content, which is a water content of dehydrated sludge during operation of the sludge treatment facility, is acquired. (3) Based on the operating flock state, the operating sludge water content, a desired flocculation flock state that is a desired flocculation flock state in a flocculation tank of the sludge treatment facility, and a desired sludge water content that is a desired water content of dehydrated sludge discharged from a sludge dehydrator of the sludge treatment facility, facility operation contents of the flocculation tank and the sludge dehydrator are selected and generated. (4) The facility operation contents are displayed on the display device.

### Advantageous Effects of Invention

According to the present invention, particularly with respect to a facility for a sludge dehydrating step and a flocculation step in a sludge treatment plant, information for performing appropriate operation management based on data obtained by digitizing experience values possessed by experienced workers in sludge treatment is displayed. Therefore, even a worker with a poor experience value can execute appropriate operation of the facility, and operation assistance and control of the sludge treatment facility for improving the efficiency of sludge treatment can be realized.

According to the present invention, when a desired flocculated flock is formed in the flocculation step, an injection amount of the polymer flocculant having a high drug unit price is appropriate, so that the cost of the drug to be used can be reduced. As a result, the cost for sludge disposal can be reduced.

Furthermore, in the sludge dehydrating step, a dehydration pressure of the sludge dehydrator is set to be appropriate, and a water content of the dehydrated sludge discharged from the sludge dehydrator can be adjusted to obtain dehydrated sludge having desired properties. As a result, it is possible to reduce the cost for sludge disposal by being suitable for conveyance and transportation after discharge (That is, it is possible to facilitate conveyance and transportation after discharge.).

### BRIEF DESCRIPTION OF DRAWINGS

FIG. 1 is a configuration diagram of a sewage sludge treatment facility operation assistance navigation system according to a first embodiment.

FIG. 2 is a configuration diagram of a sewage sludge treatment facility operation assistance navigation system according to a second embodiment.

FIG. 3 is an example of a display screen of a sewage sludge treatment facility operation assistance navigation system according to a third embodiment.

FIG. 4 is an example of a display screen of a sewage sludge treatment facility operation assistance navigation system according to a fourth embodiment.

FIG. 5 is an example of a display screen of a sewage sludge treatment facility operation assistance navigation system according to a fifth embodiment.

FIG. 6 is an example of a display screen of a sewage sludge treatment facility operation assistance navigation system according to a sixth embodiment.

FIG. 7 is an example of a display screen of a sewage sludge treatment facility operation assistance navigation system according to a seventh embodiment.

FIG. 8 is an example of a display screen of a sewage sludge treatment facility operation assistance navigation system according to an eighth embodiment.

FIG. 9 is an algorithm flow illustrating an example of use of a sewage sludge treatment facility operation assistance navigation system using an image analysis method for measured flocculated flock images according to a ninth embodiment.

FIG. 10 is an algorithm flow illustrating an example of a method of obtaining a regression line from an infrared reflectance and predicting a water content using the regression line in a sewage sludge treatment facility operation assistance navigation system according to a tenth embodiment.

FIG. 11 is a configuration diagram of a sewage sludge treatment facility operation assistance navigation system according to an eleventh embodiment.

FIG. 12 is a configuration diagram of a sewage sludge treatment facility operation assistance navigation system according to a twelfth embodiment.

FIG. 13 is a configuration diagram of a sewage sludge treatment facility operation assistance navigation system according to a thirteenth embodiment.

FIG. 14 is a configuration diagram of a sewage sludge treatment facility operation assistance navigation system according to a fourteenth embodiment.

FIG. 15 is an algorithm flow illustrating an example of a method of obtaining a regression line from infrared reflected light and predicting a water content using the regression line in a sewage sludge treatment facility operation assistance navigation system according to a fifteenth embodiment.

FIG. 16 is a diagram illustrating an example of a menu screen on a display screen of the sewage sludge treatment facility operation assistance navigation system.

FIG. 17 is a diagram illustrating an example of a screen related to status confirmation on a display screen of the sewage sludge treatment facility operation assistance navigation system.

FIG. 18 is a diagram illustrating an example of a screen related to an operation instruction on a display screen of the sewage sludge treatment facility operation assistance navigation system.

FIG. 19 is a diagram illustrating an example of a screen related to implementation content on a display screen of the sewage sludge treatment facility operation assistance navigation system.

FIG. 20 is a diagram illustrating an example of a screen related to an output of implementation content on a display screen of the sewage sludge treatment facility operation assistance navigation system.

### DESCRIPTION OF EMBODIMENTS

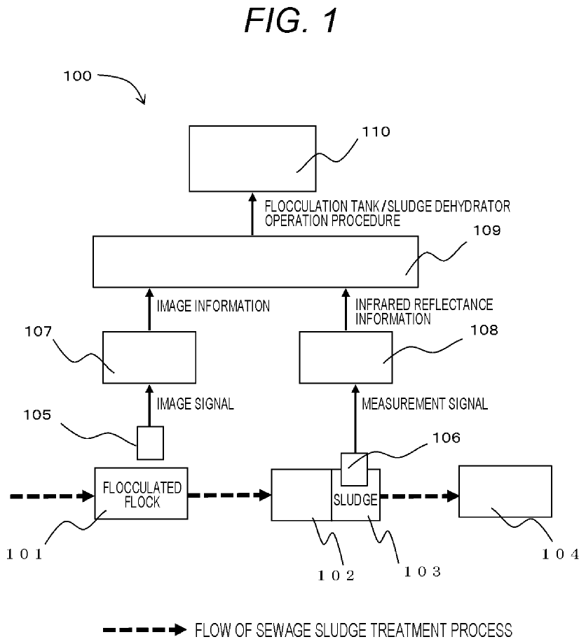

In order to solve the problems described in the section of Technical Problem, embodiments of the present invention will be described below with reference to FIGS. 1 to 20. FIG. 1 is a configuration diagram of a sewage sludge treatment facility operation assistance navigation system.

As a flow of a sewage sludge treatment process, a sewage sludge treatment facility operation assistance navigation system 100 is provided to realize operation assistance and control of a sludge treatment facility for improving the efficiency of sludge treatment in a sewage sludge treatment plant in which the sewage sludge treatment facilities of a flocculation tank 101, a sludge dehydrator, and a sludge storage tank 104 are installed.

Briefly describing the sewage sludge treatment facility described above, the flocculation tank 101 is a tank used for adding a flocculant to flocculate suspended substances in sewage water to form flocculated flocks as aggregate particles. In general, the flocculation tank 101 is provided with a stirring device, and the inside of the flocculation tank 101 is stirred by the stirring device.

The sludge dehydrator includes a sludge dehydrator dehydrating section 102 and a sludge dehydrator discharge section 103. The sludge dehydrator dehydrating section 102 constitutes a dehydrating section of the sludge dehydrator, and performs dehydration treatment of sludge. The sludge dehydrator dehydrating section 102 may be, for example, a belt press dehydrator, a screw press dehydrator, or a centrifugal dehydrator. The sludge dehydrator discharge section 103 constitutes a discharge section of the sludge dehydrator. The sludge dehydrated by the sludge dehydrator dehydrating section 102 is discharged to the outside of the sludge dehydrator via the sludge dehydrator discharge section 103. The sludge storage tank 104 is a tank used for storing sludge.

As illustrated in FIG. 1, the sewage sludge treatment facility operation assistance navigation system 100 includes an imaging device 105, an infrared measuring device 106, an imaging signal processing device 107, an infrared signal processing device 108, a computer 109 (electronic computer), and a flocculation tank/sludge dehydrator operation procedure display device 110. The flocculation tank/sludge dehydrator operation procedure display device 110 can be an appropriate display, and may be referred to as display device 110 in the present specification.

The imaging device 105 is a device having an image sensor, and converts an image of light (physical quantity) of a target object into an electrical signal (sometimes referred to as an image signal) corresponding to the intensity of the light. As an example, the imaging device 105 is provided so as to be able to image the sludge inside the flocculation tank 101, and the imaging device 105 outputs an image signal acquired by imaging to the imaging signal processing device 107. The imaging signal processing device 107 is a device that acquires image information by performing signal processing on an image signal input from the imaging device 105, and the acquired image information is output to the computer 109.

The infrared measuring device 106 is a device having an infrared sensor, and converts light (infrared ray) in an infrared region received from a target object into an electric signal (sometimes referred to as a measurement signal). The infrared measuring device 106 is appropriately provided so as to be able to measure the dehydrated sludge after the dehydration treatment by the sludge dehydrator dehydrating section 102 (provided in the sludge dehydrator discharge section 103, a flow path (for example, a conveyance path for dehydrated sludge) between the sludge dehydrator discharge section 103 and the sludge storage tank 104, the sludge storage tank 104, and the like.), and a measurement signal acquired by the infrared measuring device 106 is output to the infrared signal processing device 108. The infrared signal processing device 108 performs signal processing on the input measurement signal to acquire infrared reflectance information (infrared spectrum), and the acquired infrared reflectance information is output to the computer 109. The water content of the dehydrated sludge is determined by analyzing the infrared reflectance information.

In the present embodiment, the computer 109 includes a processor and a storage resource. The processor is configured as an arithmetic device. The storage resource can be an appropriate recording device (for example, a hard disk drive), and a plurality of data and an appropriate program for performing data processing are stored in the storage resource. The program stored in the storage resource is executed by the processor.

In the storage resource of the computer 109, a desired flocculation flock state, a desired sludge water content, and a facility operation procedure group are stored as data.

The desired flocculation flock state is data indicating a good flocculation flock state in the flocculation tank 101. That is, the desired flocculation flock state is, for example, data of a flocculation flock state that is considered to be preferable on the basis of knowledge possessed by a skilled and experienced person (That is, the data is data indicating the size and shape of the flock which is considered to be preferable based on the knowledge of the skilled and experienced person.). In this sense, a good flocculation flock state can also be said to be a targeted (desired) flocculation flock state.

The desired sludge water content is data on an excellent water content of dehydrated sludge discharged from the sludge dehydrator. As in the case of the desired flocculation flock state, the desired sludge water content is, for example, data of a water content of dehydrated sludge that is considered to be preferable based on knowledge possessed by a skilled and experienced person. In this sense, a good water content can also be said to be a target (desired) water content.

The facility operation procedure group is data including facility operation procedures of the flocculation tank 101 and the sludge dehydrator for setting the flocculation flock state in the flocculation tank 101 to an excellent flocculation flock state (that is, the desired flocculation flock state) and setting the water content of the dehydrated sludge discharged from the sludge dehydrator to an excellent water content (that is, the desired sludge water content). That is, the facility operation procedure group includes a plurality of facility operation procedures, and includes a facility operation procedure of making the flocculated flock larger than the current state and a facility operation procedure of making the flocculated flock smaller than the current state with respect to the flocculation tank 101. As an example of the facility operation procedure for making the flocculated flock larger than the current state, there is an operation procedure for injecting a flocculation agent into the flocculation tank 101. Furthermore, as an example of the facility operation procedure for making the flocculated flock smaller than the current state, there is an operation procedure for changing the rotation speed of the stirring device to increase the rotation speed. Furthermore, the facility operation procedure group includes, regarding the sludge dehydrator, a facility operation procedure of increasing the dehydration pressure to make the water content of the dehydrated sludge smaller than the current water content, and a facility operation procedure of decreasing the dehydration pressure to make the water content of the dehydrated sludge larger than the current water content. As an example of a facility operation procedure for making the water content of dehydrated sludge smaller than the current water content, there is an operation procedure for increasing the dehydration pressure by changing the dehydration pressure of the sludge dehydrator by operating a pressure adjustment mechanism (for example, a valve) provided in the sludge dehydrator. Furthermore, an example of a facility operation procedure for increasing the water content of dehydrated sludge to be higher than the current water content is an operation procedure for decreasing the dehydration pressure by changing the dehydration pressure of the sludge dehydrator by operating a pressure adjustment mechanism provided in the sludge dehydrator.

In the present embodiment, the storage resource of the computer 109 stores an operation procedure display program used to display facility operation procedures of the flocculation tank 101 and the sludge dehydrator. In the processing of the operation procedure display program, the good flocculation flock state (that is, the desired flocculation flock state) and the good water content (that is, the desired sludge water content) of the dehydrated sludge are compared with the current flocculation flock state acquired when the sewage sludge treatment facility is in operation (operating flock state) and the water content (operating sludge water content) of the dehydrated sludge to be acquired. In the operation procedure display program, a process of displaying, on the display device 110, a facility operation procedure for bringing the flocculation flock state to be acquired and the water content of the dehydrated sludge to be acquired close to the desired flocculation flock state and the desired sludge water content is performed. In the present embodiment, in the processing of the operation procedure display program, a process of selecting an appropriate facility operation procedure for bringing the flocculation flock state and the sludge water content close to the desired flocculation flock state and the desired sludge water content from the facility operation procedure stored in the storage resource is performed. For example, in a case where the current flocculated flock is smaller than the desired flocculation flock state, a facility operation procedure for making the flocculated flock larger than the current flocculated flock is selected from the facility operation procedure group, and a process of displaying the facility operation procedure on the display device 110 is performed. Furthermore, for example, in a case where the current water content is larger than the desired sludge water content, a facility operation procedure for reducing the water content of the dehydrated sludge is selected from the facility operation procedure group, and the facility operation procedure is displayed on the display device 110.

Next, an example of operation of the sewage sludge treatment facility using the sewage sludge treatment facility operation assistance navigation system 100 will be described. As illustrated in FIG. 1, in the sewage sludge treatment facility, the sewage sludge flocculation treatment is performed in the flocculation tank 101 on an upstream side, and the sludge dehydrating treatment is performed in the sewage sludge dehydrator on a downstream side of the flocculation tank 101.

During operation of the sewage sludge treatment facility, the imaging device 105 captures an image of the current flock state (the operating flock state) over time generated by addition of the flocculant, and the computer 109 acquires image information of the current flocculation flock state. Furthermore, during operation of the sewage sludge treatment facility, the current dehydrated sludge discharged from the sludge dehydrator is measured over time by the infrared measuring device 106, and the computer 109 acquires infrared reflectance information of the dehydrated sludge. Then, the computer 109 acquires the water content (operating sludge water content) of the current dehydrated sludge based on the analysis of the infrared reflectance information.

At the time of operating the sewage sludge treatment facility, the processor of the computer 109 executes the operation procedure display program to perform a process of comparing the current flocculation flock state (operating flock state) and the current water content of the dehydrated sludge (operating sludge water content) with the desired flocculation flock state and the desired sludge water content. Then, the processor outputs, to the display device 110, facility operation procedures of the flocculation tank 101 and the sludge dehydrator for bringing the current flocculation flock state and the current water content of the dehydrated sludge close to the desired flocculation flock state and the desired sludge water content, and displays the facility operation procedures. Note that in the processing of the operation content display program, comparison processing in real time is performed, and a facility operation procedure is output to the display device 110. Therefore, the latest facility operation procedure is displayed on the display device 110.

The operator of the sewage sludge treatment facility appropriately operates the flocculation tank 101 and the sludge dehydrator in accordance with the information on the latest facility operation procedure displayed on the display device 110, so that the current flocculation flock state and the current water content of the dehydrated sludge can be brought close to the desired flocculation flock state and the desired sludge water content, and the sewage sludge treatment facility can be appropriately operated.

According to the present embodiment, since the facility operation procedure of the sewage sludge treatment facility for bringing the current flocculation flock state and the current water content close to the desired flocculation flock state and the desired sludge water content is displayed, for example, even a worker with a poor experience value can assist proper facility operation and perform proper facility operation.

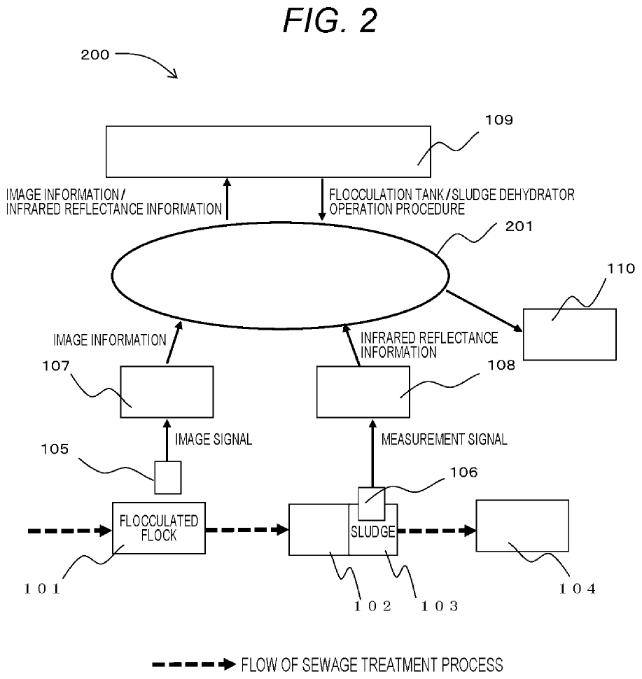

Next, a second embodiment will be described. FIG. 2 is a configuration diagram of a sewage sludge treatment facility operation assistance navigation system. Note that, in the description of the present embodiment, description overlapping with the content already described may be omitted.

In a sewage sludge treatment facility operation assistance navigation system 200 according to the second embodiment, a computer 109 is installed at a remote place different from the sewage sludge treatment plant where the sewage sludge treatment facility is provided, and the computer 109 acquires information from the sewage sludge treatment facility via a communication network 201. Furthermore, a display device 110 is installed in a sewage sludge treatment plant provided with a sewage sludge treatment facility, and the sewage sludge treatment facility operation assistance navigation system 200 performs display based on the output from the computer 109 via the communication network 201.

In the present embodiment, the computer 109 includes a processor, a storage resource, and a communication unit. The communication unit is configured as an interface for performing communication. The information output from an imaging signal processing device 107 and an infrared signal processing device 108 is input to the communication unit via the communication network 201. Furthermore, an output from the computer 109 is input to the display device 110 via the communication unit and the communication network 201.

According to the present embodiment, the computer 109 is installed in a remote place different from the sewage sludge treatment plant, and the computer 109 can be used as a distribution server for distributing information for assisting the operation of the sewage sludge treatment facility, so that the operator of the sewage sludge treatment facility can be assisted in the operation of the facility based on the information from the remote place.

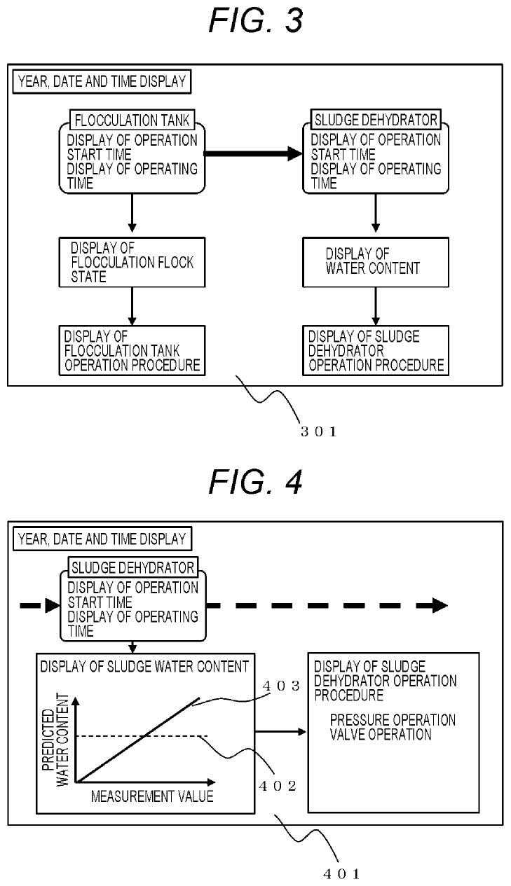

Next, a third embodiment will be described. FIG. 3 is an example of a display screen of the sewage sludge treatment facility operation assistance navigation system. Note that, in the description of the present embodiment, description overlapping with the content already described may be omitted.

According to the present embodiment, an operation procedure display device screen 301 (also referred to as display screen) of the display device 110 displays information on the current year and date, information on the flocculation tank 101, and information on the sludge dehydrator. Here, the information on the flocculation tank 101 and the information on the sludge dehydrator are classified and displayed as an example. Note that the information on the display screen 301 is displayed by executing an appropriate program. The program is stored in a storage resource and executed by a processor.

The display of the information related to the year/month/date may be displayed in an appropriate manner so that the worker can understand the information. For example, a number or a symbol is used to display the information related to the year/month/date.

As an example, the information related to the flocculation tank 101 includes information on an operation start time and an operating time of the flocculation tank 101, information on a state of the flocculated flock, and information on a facility operation procedure related to the flocculation tank 101.

The display of the operation start time and the operating time of the flocculation tank 101 may be displayed in an appropriate manner so that the operator of the sewage sludge treatment facility can understand the operation start time and the operating time of the flocculation tank 101, and numbers and symbols are used, for example.

The display of the flocculation flock state may be displayed in an appropriate manner so that the operator of the sewage sludge treatment facility can understand the current flocculation flock state, and the display of the flocculation flock state may include, for example, image information acquired over time using the imaging device 105. Furthermore, for example, the size of the flocculated flock may be displayed by a numerical value or a character, or the shape of the flocculated flock may be displayed. Furthermore, information on the desired flocculation flock state may be included.

The information on the facility operation procedure related to the flocculation tank 101 includes a facility operation procedure related to the flocculation tank 101 selected from the facility operation procedure group described in the first embodiment. Therefore, for example, a facility operation procedure of adjusting the rotation speed of the stirring device of the flocculation tank 101 to bring the flocculation flock state close to the desired flocculation flock state and a facility operation procedure of injecting the flocculation agent to bring the flocculation flock state close to the desired flocculation flock state are included. The display information of the facility operation procedure of the flocculation tank 101 may be displayed in an appropriate manner so that the operator of the sewage sludge treatment facility can understand the facility operation procedure for bringing the current flocculation flock state close to the desired flocculation flock state. Furthermore, the rotation speed of the stirring device and the injection amount of the flocculation agent for bringing the flocculation flock state into the desired flocculation flocked state may be calculated from a difference between the desired flocculation flocked state and the current flocked state by the execution of the injection amount calculation program, and the facility operation procedure generated by combining the calculated value and the facility operation procedure related to the flocculation tank 101 in the facility operation procedure group may be displayed by the execution of the operation procedure display program. For example, in a case where the current flock state is smaller than the desired flocculation flock state, the injection amount calculation program may be executed to calculate the injection amount of the main polymer flocculant for bringing the flock state into the desired flocculation flock state from the difference between the desired flocculated flock and the current flock state, and the operation procedure display program may be executed to display a facility operation procedure including the injection amount of the polymer flocculant for bringing the flock state into the desired flock state. Note that the injection amount calculation program is stored in the storage resource and executed by the processor. Furthermore, as in the case of the first embodiment, the latest facility operation procedure is displayed on the display device 110.

According to the present embodiment, a specific facility operation procedure for bringing the flocculation flock state into the desired flocculation flock state is displayed. Therefore, for example, unlike a case where the flocculated flock is monitored in the flocculation facility, an index related to the state of the flocculated flock is displayed on a monitor device, and the state of the treatment plant is simply monitored, the specific operation content is displayed. Therefore, even a worker with a poor experience value can perform appropriate operation management according to the displayed content.

The information on the sludge dehydrator includes, for example, information on an operation start time and an operating time of the sludge dehydrator, information on a water content of the dehydrated sludge, and information on a facility operation procedure related to the sludge dehydrator.

The display information of the operation start time and the operating time of the sludge dehydrator may be displayed in an appropriate manner so that the operator of the sewage sludge treatment facility can understand the display information. For example, numbers and symbols are used to display the operation start time and the operating time of the sludge dehydrator.

The information on the water content of the dehydrated sludge (In FIG. 3, it is described as display of water content.) is information on the water content of the dehydrated sludge obtained by the infrared measuring device 106. The information on the water content of the dehydrated sludge may be displayed in an appropriate manner so that the operator of the sewage sludge treatment facility can understand the current water content of the dehydrated sludge. Furthermore, information on the desired sludge water content may be included.

The information on the facility operation procedure related to the sludge dehydrator includes a facility operation procedure related to the sludge dehydrator selected from the facility operation procedures in the facility operation procedure group described in the first embodiment. Therefore, a facility operation procedure for adjusting a dehydration pressure of the sludge dehydrator to bring the sludge water content close to the desired sludge water content is included. The display information of the facility operation procedure of the sludge dehydrator may be displayed in an appropriate manner so that the operator of the sewage sludge treatment facility can understand the facility operation procedure for bringing the water content of the current dehydrated sludge close to the desired sludge water content. Furthermore, the dehydration pressure calculating program may be executed to calculate the dehydration pressure of the sludge dehydrator for achieving the desired sludge water content from a difference between the desired sludge water content and the current water content, and the operation procedure displaying program may be executed to display the facility operation procedure generated by combining the calculated dehydration pressure and the facility operation procedure related to the sludge dehydrator in the facility operation procedure group. That is, a facility operation procedure including a dehydration pressure for achieving a desired sludge water content may be displayed. Note that the dehydration pressure calculation program is stored in the storage resource and executed by the processor. Furthermore, as in the case of the first embodiment, the latest facility operation procedure is displayed on the display device 110.

According to the present embodiment, when the flocculation tank 101 and the sludge dehydrator are in operation, a facility operation procedure for adjusting a dehydration pressure of the sludge dehydrator for adjusting dehydrating sludge discharged from the sludge dehydrator to a desired sludge water content and a facility operation procedure for injecting a main polymer flocculant for bringing suspended substances in sewage into a desired flocculation flock state are displayed on the display device 110, and the facility operation procedure for injecting a polymer flocculant includes an injection amount of the main polymer flocculant.

Therefore, it is possible to present, to the operator of the sewage sludge treatment facility, a facility operation procedure relating to the injection of the main polymer flocculant in order to approximate the desired flocculation flock state and a facility operation procedure for adjusting the dehydration pressure of the sludge dehydrator in order to approximate the desired sludge water content, thereby assisting the operation of the sewage sludge treatment facility.

Next, a fourth embodiment will be described. Note that, in the description of the present embodiment, description overlapping with the content already described may be omitted.

In the present embodiment, regression line information that is data of a regression line used for predicting and calculating the water content of dehydrated sludge is stored in the storage resource. In the processing of the operation procedure display program, the predicted value of the dehydrated sludge is calculated using the regression line information stored in the storage resource, and the predicted value of the water content of the dehydrated sludge is compared with the desired sludge water content. Then, a process of displaying a facility operation procedure of the sludge dehydrator for bringing the predicted value of the water content of the dehydrated sludge close to the desired sludge water content on the display device 110 is performed.

The regression line is obtained from dehydrated sludge having a known water content. An example of a method for obtaining the regression line information will be described. In this method, dehydrated sludge having a known water content is irradiated with infrared rays, and infrared rays reflected from the dehydrated sludge are measured over time by an infrared measuring device 106 (infrared sensor). Here, data (time measurement information) acquired by time measurement is stored in the storage resource. Note that in order to obtain a regression line with high accuracy, in the present embodiment, infrared rays in a wavelength range of 1200 nm or more and 2500 nm or less are emitted. Then, in this method, infrared reflectance (infrared spectrum) is calculated from data acquired by time measurement, first order differential processing is performed on the infrared reflectance, and multivariate regression analysis is performed on a result of the first order differential processing, thereby acquiring regression line information.

A method of obtaining the regression line information will be described in more detail. As an example, the calculated infrared reflectance can be considered as spectrum data (graph) in which a vertical axis indicates reflectance (%) and a horizontal axis indicates wavelength (nm). Then, the result of performing the first derivative processing on the infrared reflectance (more specifically, a result obtained by first differentiating the reflectance value of the infrared reflectance) can be considered as a graph in which the vertical axis represents the first derivative value (dimensionless) and the horizontal axis represents the wavelength (nm). Then, a regression line is obtained by performing multivariate regression analysis on the result of the first order differential processing. Here, it is sufficient that an appropriate regression line can be obtained, and the number of variables (that is, the interval between the data points in the wavelength range) can be appropriately determined in the multivariate regression analysis. Then, the predicted value of the water content can be obtained by inputting data based on the infrared reflectance to the regression line.

As described above, in the present embodiment, in the processing of the operation procedure display program, the predicted value of the water content of the dehydrated sludge discharged over time is calculated using the infrared reflectance acquired over time and the regression line information, and the calculated predicted value of the water content of the dehydrated sludge is compared with the desired sludge water content. Here, when the regression line information is obtained, the dehydrated sludge having a known water content preferably has a water content in the range of 60 wt % or more and less than 90 wt %. Furthermore, the desired sludge water content is preferably 60 wt % or more and less than 85 wt %. As a result, a regression line with high accuracy can be acquired, and more appropriate facility operation assistance is, display of more appropriate facility operation procedure) can be performed. Note that this numerical value is an example. The desired sludge water content may be specified by a pinpoint value in addition to the specified range.

Furthermore, in the present embodiment, the sewage sludge treatment facility operation assistance navigation system can perform the display illustrated in FIG. 4. The display screen will be described with reference to FIG. 4. FIG. 4 is an example of a display screen of the sewage sludge treatment facility operation assistance navigation system.

The display screen 401 displays information on the water content of the dehydrated sludge (in FIG. 4, display of the sludge water content) and information on the facility operation procedure of the sludge dehydrator. Note that the information on the display screen 401 is displayed by executing an appropriate program. The program is stored in a storage resource and executed by a processor.

The display of the information on the water content of the dehydrated sludge can be, for example, a graph including the regression line 403. In the graph, the horizontal axis represents the measured value (water content obtained by actual measurement of the dehydrated sludge), and the vertical axis represents the predicted water content. Furthermore, in the display of the information on the water content of the dehydrated sludge, the value of the desired sludge water content 402 may be indicated by a straight line parallel to the measured value (that is, the horizontal axis). On the other hand, the display of the facility operation procedure of the sludge dehydrator includes the facility operation procedure of the sludge dehydrator based on the comparison between the predicted water content obtained by inputting the measured value obtained by measuring the current dehydrated sludge into the regression line 403 and the desired sludge water content. The operator of the sewage sludge treatment facility can operate the sludge dehydrator with reference to the sludge water content indication and the indication of the facility operation procedure of the sludge dehydrator.

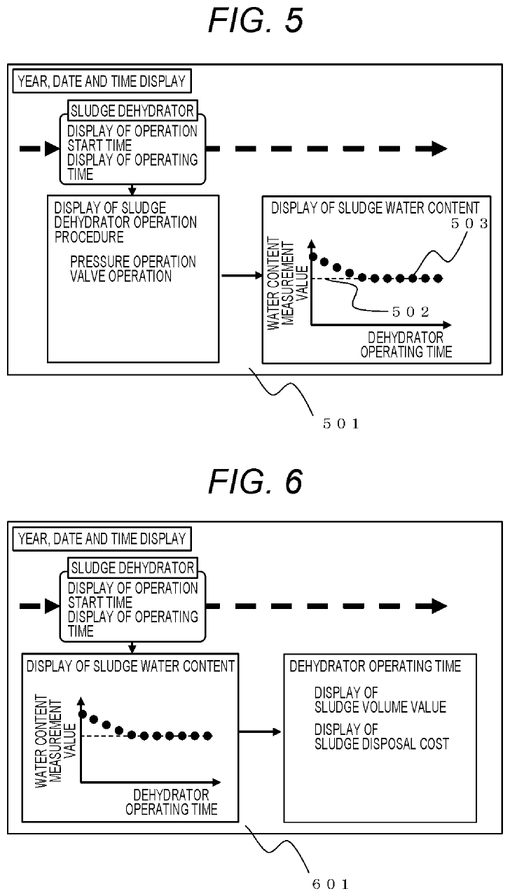

Next, a fifth embodiment will be described. FIG. 5 is an example of a display screen of the sewage sludge treatment facility operation assistance navigation system. Note that, in the description of the present embodiment, description overlapping with the content already described may be omitted.

In the present embodiment, a display screen 501 displays the transition of the water content of the dehydrated sludge when the worker operates the sludge dehydrator in accordance with the display information of the facility operation procedure of the sludge dehydrator. Specifically, the display screen 501 displays display information on a facility operation procedure of the sludge dehydrator and information on the sludge water content (in FIG. 5, display of the sludge water content). Note that the information on the display screen 501 is displayed by executing an appropriate program. The program is stored in a storage resource and executed by a processor.

Here, the sludge water content is indicated by a graph as an example, the horizontal axis indicates the dehydrator operating time, and the vertical axis indicates the water content measurement value. The dehydrator operating time on the horizontal axis indicates the transition of the operating time of the sludge dehydrator, and indicates that the time transitions from the left side to the right side of the graph. Furthermore, as an example, the water content measurement value (that is, a water content value 503) on the vertical axis can be illustrated as a plot for each predetermined time with respect to the operating time of the sludge dehydrator. Then, regarding the water content measurement value, in the graph, a plot at the left end can be indicated as a measurement value (that is, the actual measured water content of the dehydrated sludge at present), and a plot on the right side thereof can be indicated as a predicted value. In the sludge water content display, a curve obtained by curve fitting may be displayed. Furthermore, in the sludge water content display, a desired sludge water content 502 (that is, the value of the desired sludge water content 502) may be indicated by a straight line parallel to the dehydrator operating time (that is, the horizontal axis).

The transition of the water content of the dehydrated sludge when the operator operates the sludge dehydrator according to the display information of the facility operation procedure of the sludge dehydrator is obtained by implementing an appropriate program. The transition of the water content of the dehydrated sludge (change in the water content of the dehydrated sludge until reaching the desired sludge water content) can be determined by, for example, estimating the time until the water content of the dehydrated sludge converges to the desired sludge water content of 502 when the sludge dehydrator is operated in accordance with the facility operation procedure of the sludge dehydrator displayed on the display screen 501.

According to the present embodiment, when the sludge dehydrator is operated in accordance with the display information of the facility operation procedure of the sludge dehydrator, the transition (In other words, the sludge water content is changed to the desired sludge water content by operating the display information.) of the water content of the dehydrated sludge is displayed, whereby the facility operation of the operator of the sewage sludge treatment facility can be assisted.

Next, a sixth embodiment will be described. FIG. 6 is an example of a display screen of the sewage sludge treatment facility operation assistance navigation system. Note that, in the description of the present embodiment, description overlapping with the content already described may be omitted.

A display screen 601 displays the sludge water content percentage display described in the fifth embodiment, the sludge volume value as the volume value of dehydrated sludge, and the sludge disposal cost as the disposal cost of the dehydrated sludge. Note that the information on the display screen 601 is displayed by executing an appropriate program. The program is stored in a storage resource and executed by a processor.

In the present embodiment, the storage resource stores a sludge volume value calculation program, a disposal cost calculation program, and a prediction display program. Each program is executed by a processor.

The sludge volume value calculation program is a program used to calculate a sludge volume value that is a volume value of dehydrated sludge dehydrated using a sludge dehydrator. In the processing of the sludge volume value calculation display program, the sludge volume value may be estimated by an appropriate method, and the sludge volume value may be estimated based on, for example, the relationship between the dehydrator operating time (time from the start of operation to the present) and the water content of dehydrated sludge. For example, assuming that dehydrated sludge is quantitatively discharged from the sludge dehydrator, the sludge volume value may be estimated based on the dehydrator operating time. Furthermore, the sludge volume value may be estimated by adding adjustment based on the water content. Note that the sludge volume value calculation program may be used to display the calculated sludge volume value on the display device 110. The sludge volume value calculation program may be used to display the current sludge volume value on the display device 110, for example.

The disposal cost calculation program is a program used for calculating the sludge disposal cost based on the sludge volume value calculated by executing the sludge volume value calculation program. In the processing of the disposal cost calculation program, the sludge disposal cost may be estimated by an appropriate method. For example, disposal cost data in which the volume value of dehydrated sludge and the disposal cost of dehydrated sludge are associated with each other is stored in a storage resource, and the sludge disposal cost is estimated by matching the sludge volume value calculated by executing the sludge volume value calculation program with the data. Note that the disposal cost calculation program may be used to display the calculated sludge disposal cost on the display device 110. The disposal cost calculation program may be used, for example, to display the current sludge disposal cost on the display device 110.

The prediction display program is a program used to predict a sludge volume value and a sludge disposal cost with respect to an operating time of a sludge dehydrator. In the processing of the prediction display program, the prediction value of the sludge volume value and the prediction value of the sludge disposal cost may be estimated by an appropriate method.

The prediction value of the sludge volume value can be obtained as follows, for example. That is, when the sludge dehydrator is operated to dehydrate sludge having a desired sludge water content (In other words, when the sludge dehydrator is operated according to the facility operation procedure displayed on the display device 110 and the water content of the dehydrated sludge changes while converging to the desired sludge water content), data associating the operating time of the sludge dehydrator with the transition of the sludge volume value is stored in the storage resource. The sludge volume value (predicted value) at the predicted time can be obtained by matching the data with the operating time of the sludge dehydrator up to the time at which the sludge volume value is predicted.

On the other hand, as an example, the prediction value of the sludge disposal cost can be obtained by matching the prediction value of the obtained sludge volume value with the disposal cost data described above.

Furthermore, the prediction display program is a program used for causing the display device 110 to display a predicted value of the obtained sludge volume value and a predicted value of the sludge disposal cost. The manner of displaying the predicted value of the sludge volume value and the predicted value of the sludge disposal cost is not particularly limited, and for displaying these predicted values, numerical values and characters are used, for example.

In the processing in the prediction display program described above, a predicted value after an appropriate predetermined time (predicted value of sludge volume value and predicted value of sludge disposal cost) is calculated. As an example, a predicted value of the annual sludge disposal cost may be obtained. As an example, the predicted value of the annual sludge disposal cost can be obtained by the following method. For example, a predicted value of the sludge volume value per day is obtained, and the obtained sludge volume value is converted into an annual sludge volume value (That is, the predicted value×365 of the sludge volume value for one day is executed.). A predicted value of the annual sludge disposal cost is obtained by matching the obtained predicted value of the annual sludge volume value with data on the sludge disposal cost (That is, disposal cost data in which the volume value of dehydrated sludge and the disposal cost of dehydrated sludge are associated with each other). Furthermore, treatment may be performed in which a predicted value of a sludge volume value per day is obtained, a sludge disposal cost per day is obtained from the sludge volume value, and the sludge disposal cost per day is converted into an annual sludge disposal cost.

According to the present embodiment, the sludge volume value is calculated, the calculated sludge volume value is displayed on the display device 110, the sludge disposal cost is calculated, and the calculated sludge disposal cost is displayed on the display device 110, whereby the facility operation assistance of the operator of the sewage sludge treatment facility can be performed.

Furthermore, according to the present embodiment, prediction of the sludge volume value and prediction of the sludge disposal cost with respect to the operating time of the sludge dehydrator can be performed.

Furthermore, according to the present embodiment, the annual sludge disposal cost is predicted, and the predicted annual sludge disposal cost is displayed on the display device 110, whereby the facility operation assistance of the operator of the sewage sludge treatment facility can be performed.

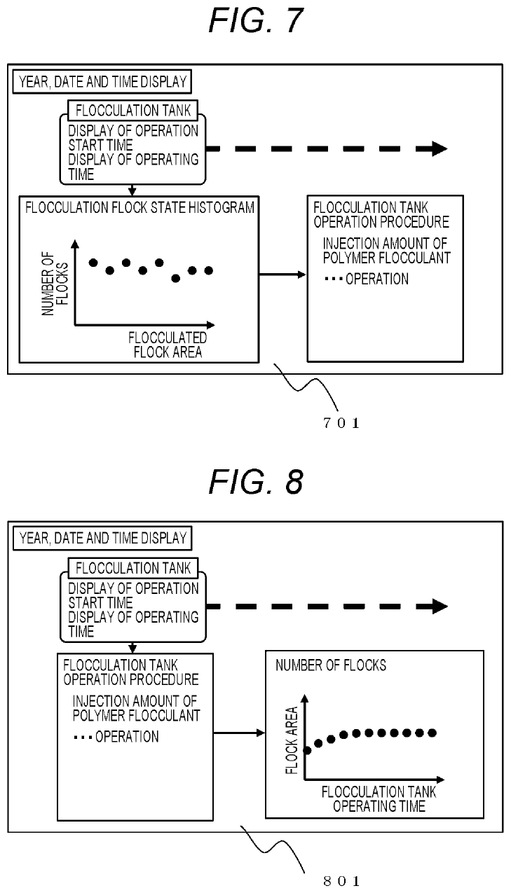

Next, a seventh embodiment will be described. FIG. 7 is an example of a display screen of the sewage sludge treatment facility operation assistance navigation system. Note that, in the description of the present embodiment, description overlapping with the content already described may be omitted.

In the present embodiment, a histogram (in the drawing, a flocculation flock state histogram) related to the state of the flocculated flock is displayed on a display screen 701. This histogram is data that is acquired by performing image processing on the image information acquired by an imaging device 105 and has the number of flocculated flocks as a frequency with the flocculated flock area, which is the area of the flocculated flocks as a grade. Note that the information on the display screen 701 is displayed by executing an appropriate program. The program is stored in a storage resource and executed by a processor.

Here, image processing for obtaining a histogram will be described. This image processing includes processing of clarifying the flocculated flock and calculating the flocculated flock area, which is the area of the flocculated flock.

Specifically, the image processing includes processing of converting image information acquired by the imaging device 105 into a monochrome image, processing of performing histogram averaging processing on the monochrome image, and processing of performing Gaussian filtering processing on the image acquired by the histogram averaging processing. That is, the image acquired by the imaging device 105 is converted into a monochrome image and flattened (That is, the contrast is increased.) by histogram averaging processing. Then, Gaussian filtering is performed on the image subjected to the histogram averaging processing (That is, processing of blurring the image and smoothly adjusting the luminance is performed.).

Moreover, the image processing includes processing of performing binarization processing on the image acquired by the Gaussian filter processing, processing of extracting a pixel region having a sewage region as a background portion in the image acquired by the binarization processing, and processing of extracting a contour of the pixel region and extracting a pixel connection region with respect to the background portion. That is, the image subjected to the Gaussian filtering processing is converted into a binary image (for example, black and white) on the basis of the binarization processing. Then, in the binary image, in order to analyze the portion of the flocculated flock, a pixel region having the sewage region as a background portion is extracted. Thereby, the portion of the flocculated flock is extracted. Moreover, a contour of the pixel region is extracted. Then, connectivity with the contour of the pixel region is determined, a portion having the same value as the pixel of the contour of the pixel region is extracted from the background portion, and a pixel connection region with respect to the background portion is extracted.

Moreover, the image processing includes processing of obtaining an integrated number of pixels obtained by integrating the number of pixels of the pixel connection region with the pixel connection region as the shape of the flocculated flock, and processing of calculating the flocculated flock area, which is the area of the flocculated flock, by converting the integrated number of pixels on the basis of the area of the image measured (acquired) by the imaging device 105. That is, in these processing, the number of pixels (integrated number of pixels) of the pixel connection region is obtained, and the flocculated flock area is converted from the integrated number of pixels in consideration of the area of the image acquired by the imaging device 105.

Then, the histogram is generated using the flocculated flock area acquired by the image processing. Here, the number of flocculated flocks can be acquired by imaging by the imaging device 105, or can be acquired as appropriate in the process of image processing, for example. Furthermore, the image processing and the processing of generating the histogram are performed by executing an appropriate program.

In the present embodiment, the processor compares a histogram acquired from image information measured with time by the imaging device 105 during operation of the sewage sludge treatment facility with a histogram relating to the desired flocculation flock state using the operation procedure display program, and displays, on the display device 110, a facility operation procedure (for example, an operation of injecting a polymer flocculant.) for bringing the state of the flocculated flock of the sewage sludge treatment facility closer to the state of the flocculated flock based on the desired flocculation flock state.

Note that, in FIG. 7, a histogram acquired by performing image processing on the image information acquired by the imaging device 105 when the sewage sludge treatment facility is in operation is displayed on the display screen 701, but for example, a histogram of a desired flocculation flock state (histogram with flocculated flock area as a grade and number of flocculated flocks as a frequency) may be displayed together on the display screen 701.

According to the present embodiment, the histogram relating to the state of the flocculated flock and the facility operation procedure (for example, an operation of injecting a polymer flocculant) for bringing the state of the flocculated flock close to the state of the flocculated flock based on the desired flocculation flock state are displayed on the display device 110, and facility operation assistance for the operator of the sewage sludge treatment facility can be performed. For example, when the flocculated flock is smaller than the desired flocculated flock, a facility operation procedure including the injection amount of the polymer flocculation agent for bringing the flocculation flock state into the desired flocculation flock state may be displayed in order to add the flocculation agent to a flocculation tank 101 to enlarge the flocculated flock (In other words, a representative flocculated flock area in the histogram is obtained.). On the other hand, for example, when in a case where flocculated flock is larger than the desired flocculated flock, a facility operation procedure of changing the rotation speed of the stirring device to increase the rotation speed may be displayed in order to increase the stirring speed of the flocculation tank 101 to reduce the flocculated flock.

Furthermore, the flocculated flocks in the image information acquired by the imaging device 105 are preferably an aggregate having a length range of 1 mm or more and 50 mm or less. As a result, it is possible to perform more appropriate facility operation assistance (that is, display of more appropriate facility operation procedure) by performing accurate image processing.

Next, an eighth embodiment will be described. FIG. 8 is an example of a display screen of the sewage sludge treatment facility operation assistance navigation system. Note that, in the description of the present embodiment, description overlapping with the content already described may be omitted.

In the present embodiment, a display screen 801 displays the transition of the flock area when the worker operates a flocculation tank 101 according to the display information of the facility operation procedure of the flocculation tank 101. Specifically, the display screen 801 displays display information of the facility operation procedure of the flocculation tank 101 and information (in FIG. 8, the number of flocks.) related to the flock area. Note that the information on the display screen 801 is displayed by executing an appropriate program. The program is stored in a storage resource and executed by a processor.

Here, the information regarding the flock area is indicated by a graph as an example, the horizontal axis indicates the operating time of the flocculation tank 101, and the vertical axis indicates the flock area. The flocculation tank operating time on the horizontal axis indicates the transition of the operating time of the flocculation tank 101, and it is illustrated that the time transitions from the left side to the right side of the graph. Furthermore, the flock area on the vertical axis can be illustrated as a plot for each predetermined time with respect to the operating time of the flocculation tank 101 as an example. Then, in the graph, a plot at the left end can be indicated as an actual measurement value (That is, the current flock area), and a plot on the right side of the left end can be indicated as a predicted value. Note that a curve obtained by curve fitting may be displayed as the information regarding the flock area. Furthermore, in the information on the flock area, the value of the flock area in the desired flocculation flock state may be indicated by a straight line parallel to the flocculation tank operating time (that is, the horizontal axis).

Furthermore, in the present embodiment, for example, the flock area can be used by calculating the area of a representative flocculated flock from the histogram relating to the state of the flocculated flock in the seventh embodiment. Furthermore, the transition of the flock area (change in the flock area until reaching the desired flocculation flock state) can be obtained, for example, by estimating the time until the flock area converges to the flock area of the desired flocculation flock state in a case where the flocculation tank 101 is operated according to the facility operation procedure of the flocculation tank 101 displayed on the display screen 801 (Here, the display of the facility operation procedure of the flocculation tank 101 can be the same as the display screen 701 and the like described in the seventh embodiment as an example.).

According to the present embodiment, by displaying the transition of the flock area (In other words, the state changes to the desired flocculation flock state by performing the operation of the display information.) when the flocculation tank 101 is operated in accordance with the display information of the facility operation procedure of the flocculation tank 101, it is possible to assist the facility operation of the worker of the sewage sludge treatment facility.

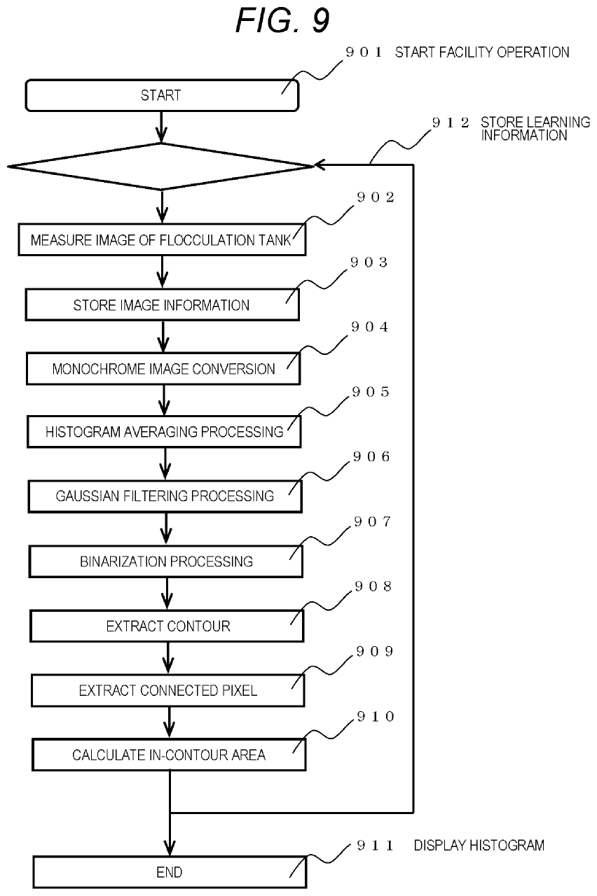

Next, a ninth embodiment will be described. FIG. 9 is an algorithm flow illustrating an example of use of a sewage sludge treatment facility operation assistance navigation system using a method of analyzing measured flocculated flock images. Note that description overlapping with the content already described may be omitted. Furthermore, the algorithm flow in FIG. 9 is processing performed by the processor executing an appropriate program.

After the operation of the sewage sludge treatment facility is started, an image of a flocculation tank 101 is measured by an imaging device 105, and the acquired image information is stored in the storage resource (processing 901 to processing 903). Then, the image processing described in the seventh embodiment is performed. That is, the monochrome image conversion processing, the histogram averaging processing, the Gaussian filtering processing, the binarization processing, the extraction of the contour, the extraction of the connected pixel region, and the calculation of the in-contour area (that is, the calculation of the flocculated flock area) are performed on the image information stored in the storage resource (processing 904 to processing 910).

Here, the above-described processing (processing 904 to processing 910) is executed by, for example, a skilled and experienced person having abundant experience values regarding the operation of the sewage sludge treatment facility. Then, the data of the state of the flocculated flock that is considered to be good for the experienced person is stored (learned) in the storage resource as the desired flocculation flock state (In this example, the histogram of the desired flocculation flock state) (processing 912).

In the subsequent processing (That is, after the processing 912 and after the learning information is stored,), the processor executes the operation procedure display program to compare the histogram (that is, the histogram acquired by performing the processing 902 to the processing 910) acquired by performing image processing on the image information acquired from the sewage sludge treatment facility with the histogram of the desired flocculation flock state acquired in the processing 912, and display the facility operation procedure of the flocculation tank 101 for bringing the current flocculation flock state closer to the desired flocculation flock state (processing 911). In this processing 911, for example, the display screen 701 on which a histogram is displayed can be displayed. By performing an operation according to the displayed facility operation procedure, it is possible to execute proper facility operation even for a worker with a poor experience value.

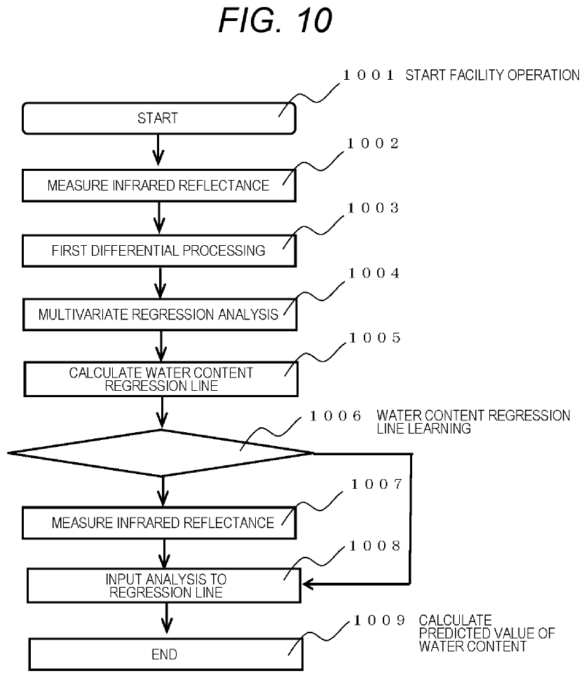

Next, a tenth embodiment will be described. FIG. 10 is an algorithm flow illustrating an example of a method of obtaining a regression line from an infrared reflectance and predicting a water content using the regression line in the sewage sludge treatment facility operation assistance navigation system. Note that description overlapping with the content already described may be omitted. Furthermore, the algorithm flow in FIG. 10 is processing performed by the processor executing an appropriate program.

After the operation of the sludge dehydrator is started (processing 1001), the regression line information described in the fourth embodiment is obtained. Specifically, the dehydrated sludge having a known water content is irradiated with infrared rays, and the infrared rays reflected from the dehydrated sludge are measured over time. Then, first differential processing is performed on the infrared reflectance calculated from the measurement information, and multivariate regression analysis is performed on the result of the first differential processing to calculate a water content regression line (processing 1002 to 1005). Then, a water content regression line calculated from dehydrated sludge having a known water content is stored (learned) in a storage resource (processing 1006).

In the subsequent processing (that is, after processing 1006,), data based on the infrared reflectance acquired when the sewage sludge treatment facility is in operation is input to the water content regression line stored (learned) in the storage resource, and a predicted value of the water content based on the water content regression line is calculated (processing 1007 to processing 1009).

Incidentally, the above-described sewage sludge treatment facility operation assistance navigation systems (100 and 200) may be provided with, for example, a temperature sensor. The temperature sensor measures and acquires data on the temperature of the dehydrated sludge discharged from the sludge dehydrator, and the data on the temperature of the dehydrated sludge may be used to select and display a facility operation procedure of the sludge dehydrator for bringing the water content of the dehydrated sludge close to the desired sludge water content in the processing of the operation procedure display program.

Here, a method of using data acquired from the temperature sensor in order to bring the water content of the dehydrated sludge discharged from the sludge dehydrator close to the desired sludge water content is not particularly limited. For example, a desired sludge temperature as data of a temperature of dehydrated sludge having a desired sludge water content may be stored in a storage resource, and in the processing of the operation procedure display program, a facility operation procedure of a sludge dehydrator for comparing a current dehydrated sludge temperature measured by a temperature sensor during operation of a sewage sludge treatment facility with a desired sludge temperature and bringing the current dehydrated sludge temperature close to the desired sludge temperature may be displayed on a display device 110. For example, when the current dehydrated sludge has a temperature lower than the desired sludge temperature, it is considered that the water content of the current dehydrated sludge is higher than the desired sludge water content. Therefore, information on the facility operation procedure for increasing the dehydration pressure of the sludge dehydrator may be displayed in the facility operation procedure group. Furthermore, for example, when the current temperature of dehydrated sludge is higher than the desired sludge temperature, it is considered that the water content of the current dehydrated sludge is lower than the desired sludge water content. Therefore, information on the facility operation procedure for reducing the dehydration pressure of the sludge dehydrator may be displayed in the facility operation procedure group. Furthermore, the dehydration pressure calculation program may be executed to calculate an appropriate dehydration pressure from the relationship between the current dehydrating sludge temperature and the desired sludge temperature, and a facility operation procedure including the calculated dehydration pressure may be displayed.

Note that, in the sewage sludge treatment facility, the position of the temperature sensor is not particularly limited as long as the temperature of the dehydrated sludge can be appropriately measured. The temperature sensor can be appropriately provided, for example, in a sludge dehydrator discharge section 103, a dehydrated sludge flow path (for example, a conveyance path for dehydrated sludge) downstream of the sludge dehydrator discharge section 103, a sludge storage tank 104, and the like. Furthermore, as an example, the measurement data of the temperature sensor is subjected to signal processing by an appropriate method and used in the processing of a computer 109. Note that the measurement data of the temperature sensor may be a temperature of sludge dehydrating itself, a temperature (air temperature) around sludge dehydrating, or both temperatures as long as the temperature relates to sludge dehydrating. A more preferable position of the temperature sensor is a position around the infrared measuring device or a position where the infrared measuring device irradiates the dehydrated sludge with infrared rays. As described above or below, the infrared measuring device irradiates the dehydrated sludge with infrared rays and calculates absorbance by reflected light, thereby obtaining a basis for water content calculation. Therefore, when the temperature of the dehydrated sludge and the air present on the infrared irradiation and reflection path is not constant, the amount of infrared rays emitted by the dehydrated sludge itself and the air itself changes. As a result, the infrared wavelength distribution (infrared spectrum) of the infrared absorbance changes depending on the temperature of the dehydrated sludge and the temperature of the air on the path, and thus the temperature needs to be considered. Note that the temperature sensor may be installed at any position of the sewage sludge treatment facility. Furthermore, the temperature indicated in the weather data may be used for the measurement of the air temperature.

As in the case of the temperature sensor described above, the sewage sludge treatment facility operation assistance navigation systems (100 and 200) may be provided with, for example, a humidity sensor. The humidity sensor may measure and acquire data on humidity around dehydrated sludge discharged from the sludge dehydrator, and in the processing of the operation procedure display program, the data on humidity of dehydrated sludge may be used to select and display a facility operation procedure of the sludge dehydrator for bringing the water content of dehydrated sludge close to the desired sludge water content.

A method of using data acquired from the humidity sensor to bring the water content of dehydrated sludge discharged from the sludge dehydrator close to the desired sludge water content is not particularly limited. For example, a desired sludge humidity as data on the humidity of dehydrated sludge having a desired sludge water content may be stored in the storage resource, and in the processing of the operation procedure display program, a facility operation procedure of the sludge dehydrator for comparing the current humidity of dehydrated sludge measured by the humidity sensor during operation of the sewage sludge treatment facility with the desired sludge humidity to bring the current humidity of dehydrated sludge close to the desired sludge humidity may be displayed on the display device 110. For example, when the current dehydrated sludge has a humidity lower than the desired sludge humidity, it is considered that the water content of the current dehydrated sludge is lower than the desired sludge water content. Therefore, information on the facility operation procedure for reducing the dehydration pressure of the sludge dehydrator may be displayed in the facility operation procedure group. Furthermore, for example, when the current dehydrated sludge has a humidity higher than the desired sludge humidity, it is considered that the water content of the current dehydrated sludge is higher than the desired sludge water content. Therefore, information on the facility operation procedure for increasing the dehydration pressure of the sludge dehydrator may be displayed in the facility operation procedure group. Furthermore, the dehydration pressure calculation program may be executed to calculate the dehydration pressure from the relationship between the current humidity of the dehydrated sludge and the desired humidity of the sludge, and a facility operation procedure including the calculated dehydration pressure may be displayed.

Note that the position of the humidity sensor is not particularly limited as long as the humidity of the dehydrated sludge can be appropriately measured. The humidity sensor can be appropriately provided, for example, in the sludge dehydrator discharge section 103, a dehydrated sludge flow path (for example, a conveyance path for dehydrated sludge) downstream of the sludge dehydrator discharge section 103, the sludge storage tank 104, and the like. Furthermore, as an example, the measurement data of the humidity sensor is subjected to signal processing by an appropriate method and used in the processing of the computer 109. Note that a more preferable position of the humidity sensor is a position around the infrared measuring device or a position where the infrared measuring device irradiates the dehydrated sludge with infrared rays. This is because if there is high-humidity air on the infrared irradiation and reflection paths, the amount of infrared rays absorbed by the air changes, and it is necessary to consider humidity. Note that if the humidity is substantially the same as the humidity of the entire sewage sludge treatment facility, data of a humidity sensor at an arbitrary position of the sewage sludge treatment facility may be substituted. Moreover, the humidity indicated by the weather data may be used.

In this manner, the facility operation procedure based on the data acquired from the temperature sensor and the humidity sensor can be displayed to assist the operation of the operator of the sewage sludge treatment facility.

The sewage sludge treatment facility operation assistance navigation systems (100 and 200) may be provided with both the temperature sensor and the humidity sensor, or may be provided with one of the temperature sensor and the humidity sensor.

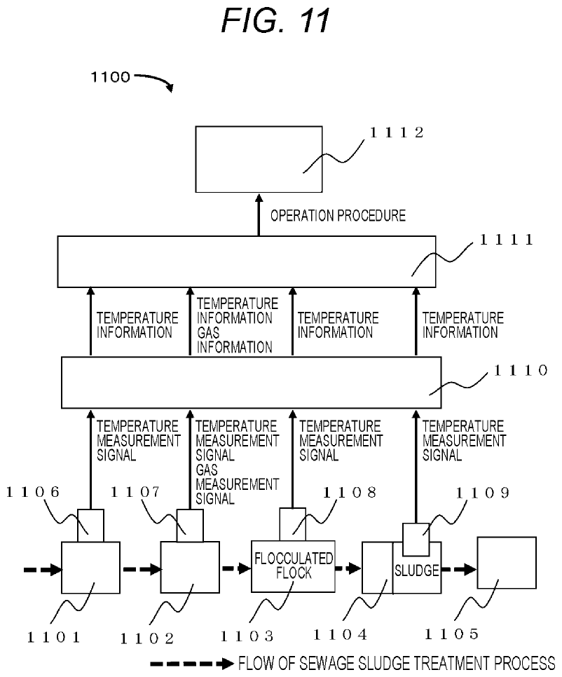

Next, an example of a measurement place by the temperature sensor will be described more specifically. FIG. 11 is a configuration diagram of a sewage sludge treatment facility operation assistance navigation system according to an eleventh embodiment. Note that, in the description of the present embodiment, description overlapping with the content already described may be omitted.

In the present embodiment, as a flow of the sewage sludge treatment process, a sewage sludge treatment facility operation assistance navigation system 1100 is provided to realize operation assistance and control of sludge treatment facilities for improving the efficiency of sludge treatment in a sewage sludge treatment plant in which the sewage sludge treatment facilities of a sludge concentration tank 1101, a concentrated sludge storage tank 1102, a flocculation tank 1103, a sludge dehydrator 1104, and a sludge storage tank 1105 are installed.

The sludge concentration tank 1101 is configured as a tank for concentrating the raw sludge supplied from the upstream side, and in the sludge concentration tank 1101, a treatment for separating water from the raw sludge to reduce the water content (As an example, the sludge is precipitated by gravity sedimentation, and the supernatant is separated to increase the concentration.) is performed. The concentrated sludge storage tank 1102 is configured as a tank for storing concentrated sludge having a reduced water content and concentrated in the sludge concentration tank 1101. In the concentrated sludge storage tank 1102, treatment for concentrating sludge may be performed. Note that, in the concentrated sludge storage tank 1102, as an example, treatment based on sedimentation may be performed as in the case of the sludge concentration tank 1101.

In the present embodiment, as illustrated in FIG. 11, the temperature sensors (1106 to 1109) are provided so as to be able to measure the temperature at a plurality of locations.

Specifically, the temperature sensor 1106 is provided so as to be able to measure the water temperature of the sludge concentration tank 1101. The temperature sensor 1107 is provided to measure the water temperature of the concentrated sludge storage tank 1102. The temperature sensor 1108 is provided so as to be able to measure the water temperature of the flocculation tank 1103. Furthermore, the temperature sensor 1109 is provided to measure the temperature of the dehydrated sludge.

Then, the temperature measurement signals acquired by the plurality of temperature sensors (1106 to 1109) are output to a signal processing device 1110. The signal processing device 1110 performs signal processing on the input temperature measurement signal to acquire temperature information, and the acquired temperature information is output to the computer 1111.

In the present embodiment, the computer 1111 can acquire information on the state of sludge entering the flocculation tank (1103) by appropriately providing a temperature sensor for measuring the water temperature in the sludge concentration tank 1101 and the concentrated sludge storage tank 1102, which are facilities of the sewage treatment process. By executing the operation procedure display program, the computer 1111 can display the facility operation contents based on the information on the state of sludge entering the flocculation tank (1103). For example, when the water temperature of the sludge concentration tank 1101 or the concentrated sludge storage tank 1102 is higher than that during normal operation, it is considered that a larger amount of gas based on fermentation (as an example, anaerobic fermentation) is generated from the sludge than that during normal operation, and the sludge in a state where fermentation has progressed (in other words, sludge having different physical properties from normal sludge) enters the flocculation tank 1103. Therefore, in this case, the injection amount of the polymer flocculant may be adjusted based on the temperature information acquired from the temperature sensor (1106 or 1107) by execution of the injection amount calculation program, and a facility operation procedure for injecting the polymer flocculant having the adjusted injection amount may be displayed on the display device 1112 by execution of the operation procedure display program. In this manner, the facility operation procedure based on the data acquired by the temperature sensor can be displayed to assist the operation of the operator of the sewage sludge treatment facility. Note that, although the example in which the temperature of the dehydrated sludge is acquired through the water temperature has been described in detail, the present invention is not limited thereto. For example, a temperature sensor may be provided at a position in contact with the dehydrated sludge to acquire the temperature of the dehydrated sludge.

Note that, as illustrated in FIG. 11, the sewage sludge treatment facility operation assistance navigation system 1100 may be appropriately provided with a gas sensor (1107) for detecting gas generated from sludge in the concentrated sludge storage tank (1102). A gas measurement signal acquired by the gas sensor (1107) is output to the signal processing device 1110. The signal processing device 1110 performs signal processing on the input gas measurement signal to acquire gas information, and the acquired gas information is output to the computer 1111. Then, as in the case of the temperature sensors (1106 and 1107) described above, the computer 1111 may adjust the injection amount of the polymer flocculant based on the gas information acquired from the gas sensor (1107) by executing the injection amount calculation program, and display, on the display device 1112, a facility operation procedure for injecting the polymer flocculant of the adjusted injection amount by executing the operation procedure display program. In this way, the gas information generated from the sludge entering the flocculation tank (1103) is incorporated, and the facility operation procedure based on the data acquired by the gas sensor (1107) is displayed, so that it is possible to assist the operation of the operator of the sewage sludge treatment facility.

Furthermore, a facility operation procedure for performing good flocculation processing for generating a desired flocculation flock state in the flocculation tank 1103 is displayed on the basis of the temperature information of the water temperature of the flocculation tank 1103 acquired by the temperature sensor 1108. For example, in a case where the water temperature of the flocculation tank 1103 is out of the reference temperature or the reference temperature range, it is conceivable that the solubility of the polymer flocculation agent is lowered and a flocculation failure occurs. Therefore, in this case, the injection amount of the polymer flocculant may be adjusted based on the temperature information acquired by the temperature sensor 1109 by executing the injection amount calculation program, and a facility operation procedure for injecting the polymer flocculant having the adjusted injection amount may be displayed on the display device 1112 by executing the operation procedure display program. Here, the reference temperature and the reference temperature range are data indicating a temperature or a temperature range in which an appropriate flocculation process for generating a desired flocculation flock state can be performed, and are stored in the storage resource.

Furthermore, in the sewage sludge treatment facility operation assistance navigation system 1100 according to the present embodiment, the same processing as the description of the temperature sensor in the case of the sewage sludge treatment facility operation assistance navigation systems (100 and 200) is performed. That is, by the processing of the operation procedure display program, a facility operation procedure for bringing the water content of the dehydrated sludge close to the desired sludge water content is displayed on the display device 1112 based on the temperature information of the dehydrated sludge acquired by the temperature sensor 1109.

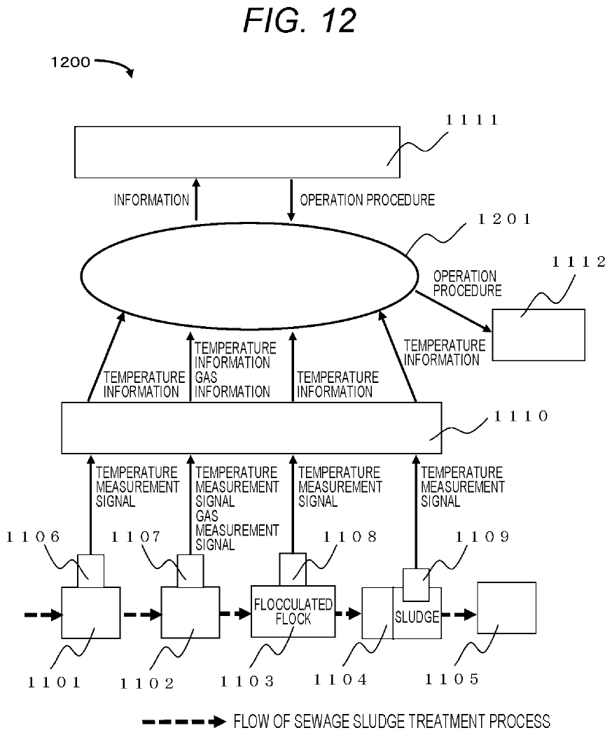

Next, a sewage sludge treatment facility operation assistance navigation system 1200 according to a twelfth embodiment will be described. FIG. 12 is a configuration diagram of a sewage sludge treatment facility operation assistance navigation system. Note that, in the description of the present embodiment, description overlapping with the content already described may be omitted.

In a sewage sludge treatment facility operation assistance navigation system 1200 according to the twelfth embodiment, a computer 1111 is installed at a remote place different from the sewage sludge treatment plant where the sewage sludge treatment facility is provided, and the computer 1111 acquires information from the sewage sludge treatment facility via a communication network 1201. Furthermore, a display device 1112 is installed in a sewage sludge treatment plant provided with a sewage sludge treatment facility, and the sewage sludge treatment facility operation assistance navigation system 1200 performs display based on the output from the computer 1111 via the communication network 1201.

In the present embodiment, the computer 1111 includes a processor, a storage resource, and a communication unit. The communication unit is configured as an interface for performing communication. Information output from a signal processing device 1110 is input to the communication unit via the communication network 1201. Furthermore, the output from the computer 1111 is input to the display device 1112 via the communication unit and the communication network 1201.

According to the present embodiment, the computer 1111 is installed in a remote place different from the sewage sludge treatment plant, and the computer 1111 can be used as a distribution server for distributing information for assisting the operation of the sewage sludge treatment facility, so that the operator of the sewage sludge treatment facility can be assisted in the operation of the facility based on the information from the remote place.

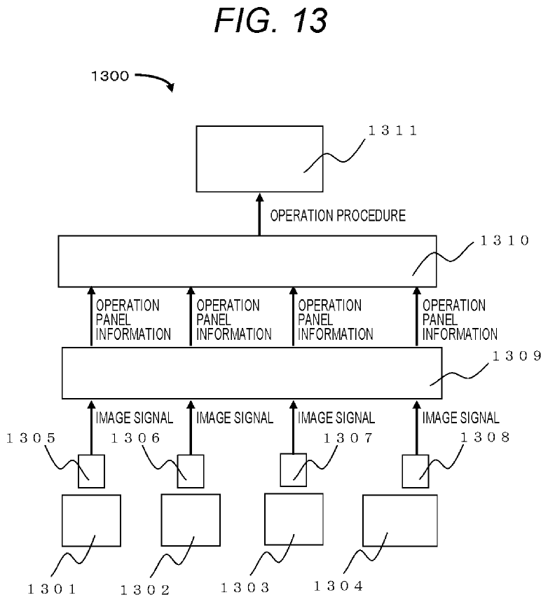

Next, a sewage sludge treatment facility operation assistance navigation system 1300 according to a thirteenth embodiment will be described. FIG. 13 is a configuration diagram of a sewage sludge treatment facility operation assistance navigation system. Note that, in the description of the present embodiment, description overlapping with the content already described may be omitted.

In the present embodiment, as illustrated in FIG. 13, the sewage sludge treatment facility operation assistance navigation system 1300 includes a plurality of image capturing machines (1305 to 1308) for capturing an operation panel of the sewage sludge treatment facility. Specifically, the image capturing machine 1305 is provided so as to be able to capture an image of an operation panel 1301 of the sludge concentration tank. The image capturing machine 1306 is provided so as to be able to capture an image of an operation panel 1302 of the concentrated sludge storage tank. The image capturing machine 1307 is provided so as to be able to capture an image of an operation panel 1303 of the flocculation tank. The image capturing machine 1308 is configured to capture an image of an operation panel 1308 of the sludge dehydrator.

Then, the image signals acquired by the plurality of image capturing machines (1305 to 1308) are output to a signal processing device 1309. The signal processing device 1309 performs signal processing on the input image signal to acquire operation panel information, and the acquired operation panel information is output to a computer 1310.

In the present embodiment, by appropriately providing an image capturing device that captures an image of an operation panel for operating the sludge concentration tank 1301, the concentrated sludge storage tank 1302, the flocculation tank 1303, and the sludge dehydrator 1304, a state indicated by an instrument of the operation panel can be acquired by image recognition from the captured image, and an appropriate facility operation procedure can be displayed on a display device 1311. Here, the image recognition can be an appropriate matching process. That is, image data obtained by capturing an image of the desired floc state and the state indicated by the instrument of the operation panel at the desired sludge water content is recorded in the storage resource, and the image data is compared with image data obtained by capturing an image of the state indicated by the current instrument by executing an appropriate image recognition program. Note that the image recognition program is stored in a storage resource and executed by a processor. Then, the computer 1310 executes the operation procedure display program to appropriately select, based on the result of the image recognition, a facility operation procedure for bringing the state close to the desired flock state and the state indicated by the meter at the desired sludge water content from the facility operation procedure group, or appropriately generate a facility operation procedure including the injection amount of the polymer flocculant and the dehydration pressure of the sludge dehydrator, and display the facility operation procedure on the display device 1311. As described above, in the present embodiment, the facility operation procedure based on the meter data on the operation panel can be displayed to assist the operation of the operator of the sewage sludge treatment facility.

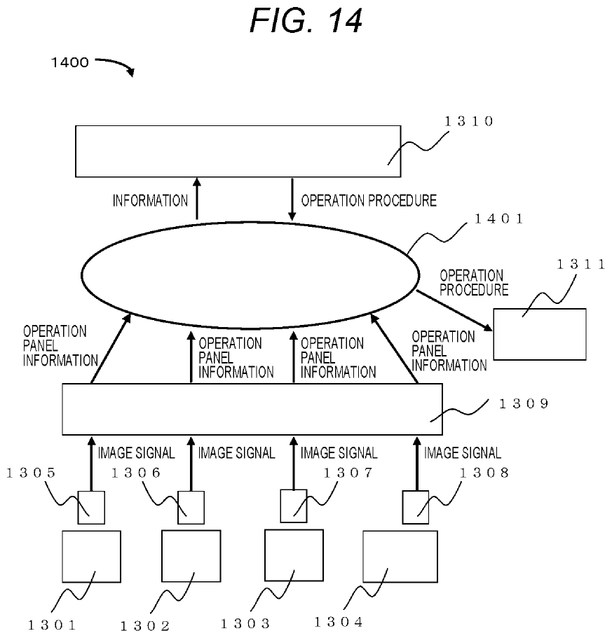

Next, a sewage sludge treatment facility operation assistance navigation system 1400 according to a fourteenth embodiment will be described. FIG. 14 is a configuration diagram of a sewage sludge treatment facility operation assistance navigation system. Note that, in the description of the present embodiment, description overlapping with the content already described may be omitted.

In the sewage sludge treatment facility operation assistance navigation system 1400 according to the fourteenth embodiment, a computer 1310 is installed at a remote place different from the sewage sludge treatment plant where the sewage sludge treatment facility is provided, and the computer 1310 acquires information from the sewage sludge treatment facility via a communication network 1401. Furthermore, a display device 1311 is installed in a sewage sludge treatment plant provided with a sewage sludge treatment facility, and the sewage sludge treatment facility operation assistance navigation system 1400 performs display based on the output from the computer 1310 via the communication network 1401.

In the present embodiment, the computer 1310 includes a processor, a storage resource, and a communication unit. The communication unit is configured as an interface for performing communication. Information output from a signal processing device 1309 is input to the communication unit via the communication network 1401. Furthermore, the output from the computer 1310 is input to the display device 1311 via the communication unit and the communication network 1401.

According to the present embodiment, the computer 1310 is installed in a remote place different from the sewage sludge treatment plant, and the computer 1310 can be used as a distribution server for distributing information for assisting the operation of the sewage sludge treatment facility, so that the operator of the sewage sludge treatment facility can be assisted in the operation of the facility based on the information from the remote place.

As described above, the present invention is not limited to the above-described embodiments, and includes various modifications. For example, the above-described embodiments have been described in detail for better understanding of the present invention, and are not necessarily limited to those having all the configurations of the description.

The sewage sludge treatment facility operation assistance navigation systems (1100, 1200, 1300, and 1400) may also perform processing using the imaging device 105, the infrared measuring device 106, the humidity sensor, and the like, as described in the case of the sewage sludge treatment facility operation assistance navigation systems (100 and 200).

The position of the infrared measuring device 106 described above is not particularly limited as long as the dehydrated sludge can be appropriately measured. The infrared measuring device 106 can be appropriately provided, for example, in the sludge dehydrator discharge section 103, a dehydrated sludge flow path (for example, a conveyance path for dehydrated sludge) downstream of the sludge dehydrator discharge section 103, the sludge storage tank 104, and the like.

The position of the imaging device 105 described above is not particularly limited as long as the flocculated flock in the flocculation tank 101 can be appropriately measured. The imaging device 105 can be appropriately provided, for example, at a position where the flocculated flock in the flocculation tank 101 can be measured, inside the flocculation tank 101, outside the flocculation tank 101, a flow path of the sludge on the downstream side of the flocculation tank 101, and the like.

In the above embodiment, the imaging signal processing device 107 and the infrared signal processing device 108 perform the signal processing of the acquired data. However, for example, the imaging signal processing device 107 and the infrared signal processing device 108 may be configured as a part of a computer, and the signal processing of the data acquired by the computer may be performed. Furthermore, the imaging signal processing device 107 and the infrared signal processing device 108 may be omitted, a program for performing the signal processing may be stored in a storage resource of a computer, and the signal processing may be performed by execution of the program by a processor. Similarly, the signal processing devices (1110 and 1309) may be configured as a part of the computer. The signal processing devices (1110 and 1309) may be omitted, and the computer may perform the signal processing.

As an example of the processor, a CPU can be considered, but another semiconductor device (for example, GPU) may be used as long as it is a subject that executes predetermined processing.

As an example, the storage resources are stored in a hard disk drive (HDD), but the storage resource may be an appropriate recording device. The storage resource may be, for example, a solid state drive (SSD).

A fifteenth embodiment will be described. Note that the same description as above may be omitted. In a fifteenth embodiment, an embodiment of a sewage sludge treatment facility operation assistance navigation system that calculates a water content prediction value, which is a prediction value of a water content, by a calculation method different from that in the fourth embodiment will be described. In this embodiment, the method of calculating the water content is different, and the method of using the water content in the sewage sludge treatment facility operation assistance navigation system is the same as that in the fourth embodiment, and thus the description of the method of use may be omitted.

In the present embodiment, regression line information that is regression line data used for predicting and calculating the water content of dehydrated sludge is stored in the storage resource. In the processing of the operation procedure display program, the predicted value of the dehydrated sludge is calculated using the regression line information stored in the storage resource, and the predicted value of the water content of the dehydrated sludge is compared with the desired sludge water content. Then, a process of displaying a facility operation procedure of the sludge dehydrator for bringing the predicted value of the water content of the dehydrated sludge close to the desired sludge water content on the display device 110 is performed.

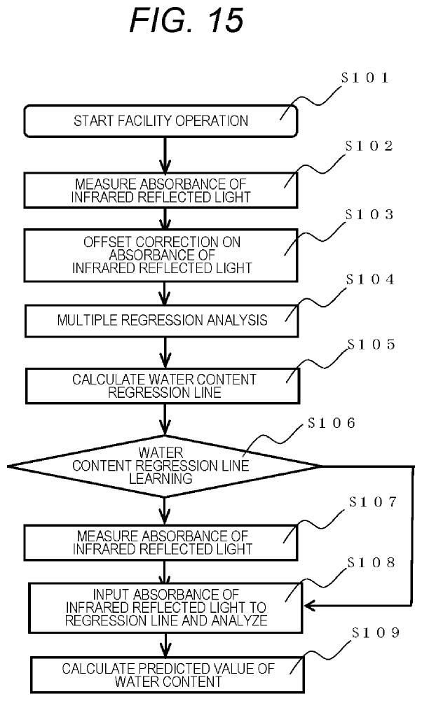

The regression line is obtained from dehydrated sludge having a known water content. Here, an example of a method of obtaining the regression line information will be described with reference to FIG. 15. FIG. 15 is a flowchart illustrating an example of an analysis flow for calculating the water content of dehydrated sludge. In this method, the dehydrated sludge having a known water content is irradiated with infrared rays after the start of the facility operation (S101), and the infrared rays reflected from the dehydrated sludge are measured over time by an infrared measuring device (infrared sensor). Here, data (time measurement information) acquired by time measurement is stored in the storage resource. Note that in order to obtain a regression line with high accuracy, in the present embodiment, infrared rays in a wavelength range of 1200 nm or more and 2500 nm or less are emitted. Then, in this method, the absorbance (infrared spectrum) of the infrared reflected light is calculated from the data acquired by the time measurement (S102), smoothing processing is performed on the absorbance of the infrared reflected light, and offset correction (base line correction) is performed on the result of the smoothing processing (S103). Thereafter, in this method, multivariate regression analysis (in this example, multiple regression analysis) is executed (S104), regression line information is acquired (S105), and the calculated regression line information of the water content is stored (learned) in the storage resource (S106). Note that the regression line information may be, for example, a regression coefficient or a correction coefficient, but may be another coefficient as long as the coefficient defines a regression line (which does not actually need to be a linear function based on the definition of statistics). Furthermore, in a case where the regression line can be expressed by a plurality of functions, the regression line may be an identifier given to a function indicating which function is applied, or may be a weighting coefficient for adding results of a plurality of functions.

A method of obtaining the regression line information will be described in more detail. As an example, the calculated absorbance of the infrared reflected light can be considered as spectrum data (graph) in which the vertical axis indicates the absorbance (arbitrary unit) and the horizontal axis indicates the wavelength (nm). Then, smoothing processing is performed on the absorbance of the infrared reflected light, an offset correction (baseline correction) is performed on the result of the smoothing processing, and a multivariate regression analysis is performed on the result, whereby a regression line is obtained. Here, it is sufficient that an appropriate regression line can be obtained, and the number of variables (that is, the number of data points in the wavelength range) can be appropriately determined in the multivariate regression analysis. Then, the predicted value of the water content can be obtained by inputting data based on the absorbance of the infrared reflected light to the regression line.

As described above, in the present embodiment, in the processing of the operation procedure display program, the predicted value of the water content of the dehydrated sludge to be discharged over time is calculated (S107 to S109) using the absorbance of the infrared reflected light acquired over time and the regression line information, and the calculated predicted value of the water content of the dehydrated sludge is compared with the desired sludge water content. Here, when the regression line information is obtained, the dehydrated sludge having a known water content preferably has a water content in the range of 60 wt % or more and less than 90 wt %. Furthermore, the desired sludge water content is preferably 60 wt % or more and less than 85 wt %. As a result, a regression line with high accuracy can be acquired, and more appropriate facility operation assistance (that is, display of more appropriate facility operation procedure) can be performed. Note that, as described above, the desired sludge water content may be specified as a pinpoint value in addition to the specified range.

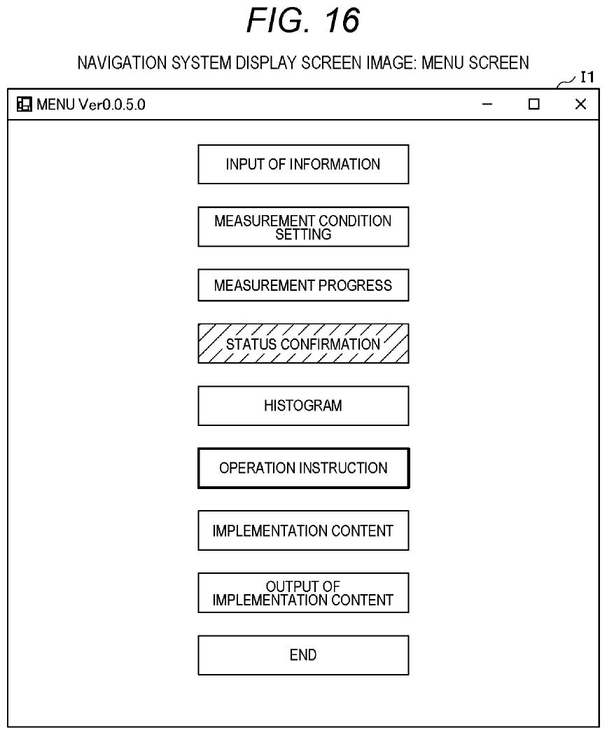

Next, details of the contents displayed on the execution screen of the sewage sludge treatment facility operation assistance navigation system will be described with reference to FIGS. 16 to 20.

FIG. 16 is an example of a menu screen. In the present embodiment, a menu screen I1 illustrated in FIG. 16 is displayed on the display device by the processor executing the program of the sewage sludge treatment facility operation assistance navigation system. As illustrated in FIG. 16, the menu screen I1 includes a plurality of items, and the operator of the sewage treatment plant can appropriately select and determine an item to be referred to. Next, contents of the items displayed on the menu screen I1 will be sequentially described.

In the item of “input information”, the worker of the sewage treatment plant can register registration information which is information to be registered in the system. Here, examples of the registration information include information such as an operator, temperature, and humidity. The registration method may be a mode that can be selected in a pull-down format or a mode that allows an operator to directly input information. Furthermore, data acquired from the sensor may be automatically input (registered) with respect to numerical data such as temperature and humidity. Furthermore, weather data may be acquired as appropriate, and numerical data such as temperature and humidity based on the weather data may be automatically input (registered). As an example, the weather data may be acquired from the outside via a network.

Here, in addition to the imaging device of the temperature, the humidity, the flocculated flock, and the infrared measuring device of the water content, the sensor includes a measurement instrument that measures a flocculation agent addition flow rate and a concentration in the flocculation tank, a rotation speed of the stirrer, a flow rate of sludge and a concentration of sludge, a value related to a squeezing pressure of sludge in the dehydrator, and the like. For example, there are sensors that measure a centrifugal force in the case of a centrifugal dehydrator, a pressure in the case of a pressure dehydrator, adjustment of a tension pressure of a filter cloth in the case of a belt press dehydrator, a rotation speed of sludge in the case of a multiple disc dehydrator, and a rotation speed of a screw in the case of a screw press dehydrator, but the sensors are not limited to these exemplified measurement sensors (measurement instruments). Note that specific examples of some of the exemplified sensors will be described later.

The item “measurement condition setting” is an item mainly intended for a program creator of a navigation system. The “measurement condition setting” stores an algorithm for analyzing an image of flocculated flocks in a flocculation tank and an algorithm for multiple regression analysis for calculating the water content of dry sludge (dehydrated sludge).

In the flocculated flock analysis algorithm, an image region, histogram flattening, a filter method, a binarization threshold value, and the like can be set. Furthermore, the range of the flocculated flock size to be analyzed may be designated, the diameter or area of the range of the flocculated flock to be analyzed, the range of the average flock diameter, the range of the flock number, and the like may be set. For example, by setting the size of the flocculated flock, the generation state of the flocculated flock having a desired size range can be easily checked.

On the other hand, in the calculation of the water content, it is possible to input (set) a target water content range, measurement conditions (number of scans, gain setting, presence or absence of smoothing, measurement speed, and the like) of a sensor for measuring the water content, calibration data, a regression coefficient for converting infrared optical information into the water content, a correction coefficient, and the like.

In addition, the state of the flocculated flocks and the measurement of the water content can be set by inputting the processing (update) interval of the real-time data. Furthermore, it is possible to input and set the maximum display time of data related to these measurements. Then, by pressing a home button, it is possible to return to the menu screen.

The items of “measurement progress” and “histogram” are items mainly intended for the program creator of the navigation system, similarly to the item of “measurement condition setting”. Data related to the measurement progress may be stored in the item of “measurement progress”. Furthermore, data and the like used for histogram processing may be stored in the item of “histogram”. However, the screen display of these items may be omitted.

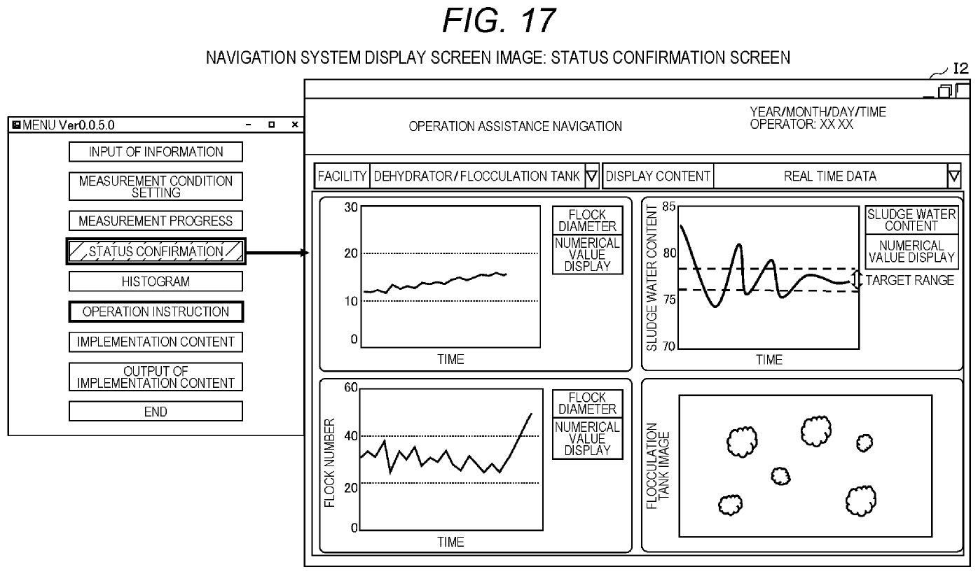

Next, the item of “status confirmation” will be described. The item “status confirmation” mainly relates to a screen targeted for workers in a sewage treatment plant. An example of the “status confirmation”, that is, an example of this screen will be described with reference to FIG. 17. FIG. 17 illustrates an example of a screen related to status confirmation.

By using a screen I2 that can be confirmed from the item of “status confirmation”, the worker can grasp the current status of the size and number of flocs and the water content by numerical values. Furthermore, the image of the flocculated flocks can be grasped using the screen I2. Here, regarding data that can be displayed as a numerical value, information (As illustrated in FIG. 17, for example, a graph in which a numerical value and a time axis are associated with each other.) indicating a change in the numerical value based on the lapse of time may be displayed. Note that numerical data of infrared spectrum information for calculating the number and diameter of the current flocs, water content, and water content can be stored and output.

Furthermore, an upper limit value and a lower limit value may be set for data that can be displayed as numerical values, and a warning may be issued when the current value deviates from the set value. As illustrated in FIG. 17, for example, an upper limit value and a lower limit value of the water content of the dried sludge (dehydrated sludge) may be set, and when the water content deviates from the set values (that is, when a target range is deviated), a number or a display frame may be flickered in red to give a warning of abnormality. Note that the warning may be issued by an appropriate means, and the mode of the warning is not particularly limited as long as the worker or the like can grasp the warning. For example, the warning may be issued by sounding a warning sound by a buzzer or the like.

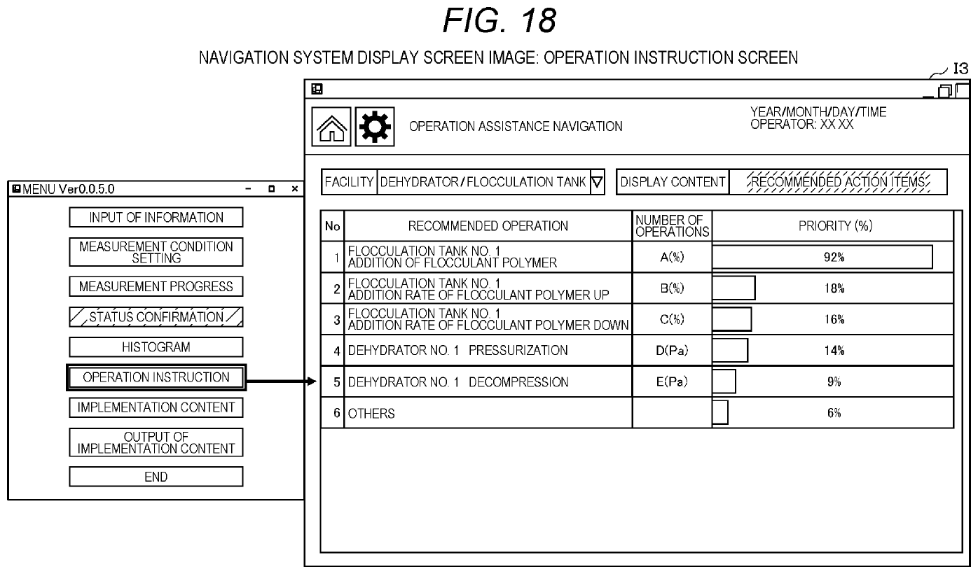

Next, the item “operation instruction” will be described. The item “operation instruction” relates to a screen that displays contents of recommended facility operation. An example of the “operation instruction”, that is, an example of the screen will be described with reference to FIG. 18. FIG. 18 illustrates an example of a screen related to an operation instruction.

On a screen I3 related to “operation instruction”, recommended facility operation contents are displayed from the current size and number of flocculated flocks and the current water content of the dried sludge based on the analysis result of the operation history of the facility, the past size and number of flocculated flocks, and the past water content of the dried sludge (dehydrated sludge).

Here, the facility operation contents are associated with a priority that is a value for recommending the operation content, and in a case where a plurality of facility operation contents are displayed, the facility operation contents are displayed on the screen I3 with priority based on the priority. Moreover, the number of operations (operation quantity) is also displayed on the screen I3.

Examples of equipment to be subjected to the recommended operation include a flocculation tank and a dehydrator. Here, in a case where a plurality of flocculation tanks and dehydrators are provided, the recommended operation is displayed in a distinguished mode as to which facility to operate. For example, in a case where there are a plurality of facilities such as a flocculation tank and a dehydrator, a display including information indicating which facility is an operation target is performed. Therefore, it is possible to easily grasp which facility should be operated by referring to the display.

A case of using a screw press type dehydrator will be described. In this case, for the flocculation tank, an operation of adding an inorganic flocculant, a polymer flocculant, or the like, or an operation of increasing or decreasing the addition rate can be cited as the facility operation content. On the other hand, for the dehydrator, an operation to increase or decrease the screw rotation speed and an operation such as a degree of pressurization or a degree of depressurization for dehydrating sludge can be cited as the facility operation content. Then, the number operated in each operation is displayed in association with each other. Furthermore, the priorities of the facility operation contents are displayed together, and the facility operation contents are displayed in order of priority.

After performing the recommended operation content on the sewage treatment facility, the operator of the sewage treatment plant can input the performed operation content. There may be provided a convenient mechanism that transitions to an input screen for “implementation content” to be described later by selecting any of the items (No, recommended operation, quantity of operations, priority) in the row of the actual operation details (that is, the content of the operation with the highest priority) among the facility operation details on the screen. That is, the screen I3 may be a screen for inputting a content actually operated using a mouse or the like, or an input of an execution content may be performed on the screen I3. With this configuration, it is possible to easily input the implementation content using the screen I3. On the other hand, the menu screen I2 may be output by selecting the home button or the like, and the “implementation content” may be selected from the menu screen I2 to input the operation content.

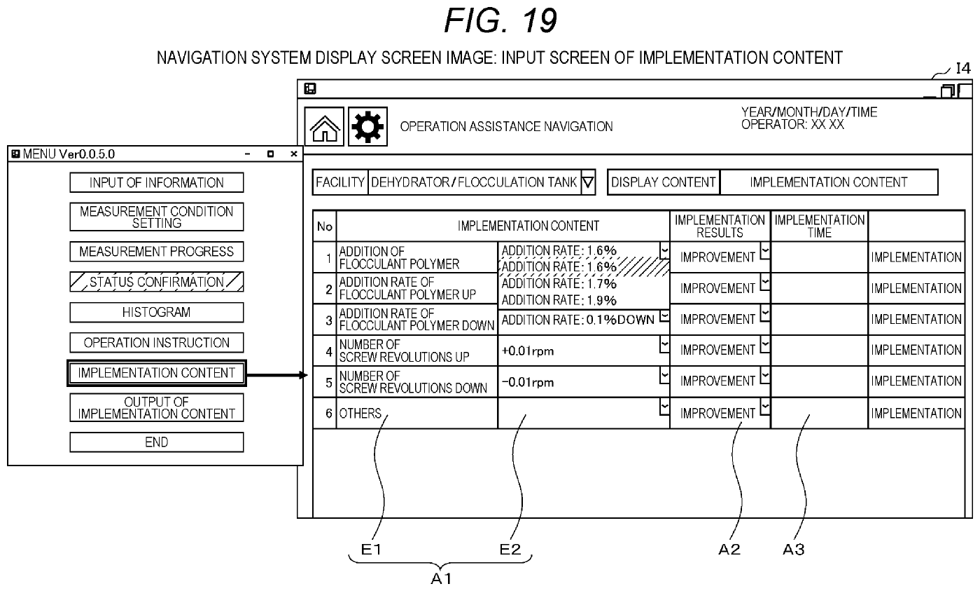

Next, the item of “implementation content” will be described. The item “implementation content” relates to an input screen of the actual facility operation content. An example of the screen of “implementation content” will be described with reference to FIG. 19. FIG. 19 illustrates an example of a screen according to the implementation content.

As illustrated in FIG. 19, a screen I4 on which “implementation content” is input includes a first area A1, a second area A2, and a third area A3. The first area A1 is an area for inputting the contents of implementation (that is, the actual facility operation content). The first area A1 includes an input field E1 related to the operation item and an input field E2 related to the operation quantity. The second area A2 is an area for inputting the quality of the result of the implementation content. The third area A3 is an area for inputting a time of implementation (implementation time). That is, the display device displays a screen including the first area A1 for inputting or selecting the actual facility operation contents by the user, the second area A2 for inputting or selecting the quality of the result of implementing the facility operation content, and the third area A3 for inputting the implementation time. In other words, the processor causes the display device to display a screen including the first area A1 for inputting or selecting the actual facility operation contents by the user, the second area A2 for inputting or selecting the quality of the result of implementing the facility operation contents, and the third area A3 for inputting the implementation time. Note that, in FIG. 19, the first area A1, the second area A2, and the third area A3 are arranged side by side as an example, but the arrangement of the respective areas (A1 to A3) on the screen I4 is not particularly limited. Then, as described above, when the content actually operated on the screen I3 of the “operation instruction” is selected, the content related to this operation may be automatically input and the input content may be displayed on the input screen I4 of the implementation content (specifically, in the first area A1). For example, in a case where an operation related to “addition of flocculant polymer” illustrated in FIG. 18 is selected, the content of this operation may be automatically input and displayed in the input field E1 of the first area A1, and at the same time, the operation quantity may be automatically input and displayed in the input field E2 of the first area A1. That is, the processor may receive specification of the facility operation contents on the screen I3, specify the operation item and the operation quantity related to the received facility operation content, and input the specified operation item and operation quantity to the first area A1. On the other hand, the input screen I4 of “implementation content” may be a screen for inputting the implementation content by the user.

In the screen I4 of (specifically, in the input field E2 of the first area A1,) of “implementation content”, the number of significant digits or the like operated is not particularly limited as long as it is appropriate, and can be appropriately determined, for example, according to the state of the sewage treatment plant (for example, in consideration of the scale of the sewage treatment plant and the treatment capacity per hour,). For example, the addition rate of the flocculant and the number of screw revolutions are appropriately determined according to the state of the sewage treatment plant, but as an example, a mechanism capable of displaying fine values up to about the second decimal place can be used.

As a result of performing the facility operation content, the operator of the sewage treatment plant can input results (implementation results) such as whether the flock aggregation state and the water content have been improved, whether there is no change, and whether further addition is necessary, to the second area A2. Note that an automatic input may be performed, and the processor may determine the quality of the implementation result from the transition of the flocculated flocks and the transition of the water content after implementation, and input the implementation result to the second area A2. Furthermore, the implementation time is input to the third area A3. The time may be automatically recorded along with the automatic input of the implementation content, or may be input by an operator of the sewage treatment plant.

A mechanism for selecting and inputting the operated quantity and execution result in a pull-down format may be provided (that is, regarding the input to the input field E2 of the first area A1 and the input to the second area A2). For example, a plurality of items indicating different addition rates may be prepared for the manipulated quantity of “addition of flocculant polymer”, and the value of the manipulated quantity may be input by appropriately selecting from these items. Furthermore, for example, items of “improvement”, “no change”, and “addition necessary” are prepared for the implementation result, and the implementation result may be input by appropriately selecting from these items. By adopting a pull-down type input method for the number of operations and further adopting a mechanism for manually inputting the operation content, it is possible to manually input the content performed by the determination by the operator of the sewage treatment plant without using the automatic input based on the selection of the facility operation content.

Data (for example, data on the content of the performed thereof) facility operation and the result input to the “implementation content” (screen I4) can be used for program setting and the like as an example, as described below.

When it is confirmed on the screen of “status confirmation” that the desired size and number of flocculated flocks and the water content of the dried sludge have been reached after the recommended operation is selected in the item of “operation instruction”, the program is set to increase the degree of recommendation (that is, the priority) when the recommended operation is displayed in the item of “operation instruction” (referred to as pattern 1) since the selected operation is appropriate. When the desired size and number of flocculated flocks, the water content of the dried sludge are not reached as a result of selecting from the recommended operation and performing the recommended operation, and the size and number of flocculated flocks, and the water content of the dried sludge are deteriorated as compared to before the recommended operation is performed as a result of selection from the recommended operation, the program is set to lower the degree of recommendation (pattern 2). When the desired size and number of flocculated flocks and the water content of the dried sludge have not been reached as a result of selecting from the recommended operation and performing the recommended operation, the program is set to lower the degree of recommendation when there is no change compared to before performing the recommended operation, but decrease the lowering width so as to be smaller than the lowering width of the setting in which the degree of recommended is lowered in pattern 2 (referred to as pattern 3). Note that the increase and decrease of the degree of recommendation herein are appropriately set by the developer. In this way, the display content of the recommended operation in the item “operation instruction”, the result of the recommended operation, the size and number of flocculated flocks, and the water content of the dried sludge are constructed as a database, and learning and analysis are repeated, so that the display content can be used to improve the accuracy of the operation instruction content (That is, more accurate facility operation contents are output.). That is, by executing the step of acquiring data on the flocculated flock and the dehydrated sludge after the recommended operation is performed, the step of evaluating the quality of the recommended operation using the acquired data, and the step of varying the priority that is a value for recommending the recommended operation according to the evaluation result, the accuracy of the operation instruction content can be improved, and more accurate recommended operation can be output.

Furthermore, the accuracy of the value of the operation quantity displayed on the screen I3 of “operation instruction” may be improved using the acquired data. For example, the accuracy of the value of the operation quantity may be improved by performing a process in which the value of the operation quantity for the corresponding facility operation contents is not changed when it is confirmed that there is an improvement from the implementation result (that is, the input contents of the second area A2) on the screen I4 of “implementation content”, and the value of the operation quantity for the corresponding facility operation contents is changed when it is confirmed that there is no improvement from the implementation result. In this way, it is possible to improve the accuracy of the value of the quantity to be operated from the quantity of operations performed (the quantity input on the input screen of the implementation content).

Furthermore, similarly, when it is confirmed on the screen I2 of “status confirmation” that the data on the water content of the flocculated flock and the sludge has reached a desired numerical value, the value of the operation quantity may not be changed, and when it cannot be confirmed that the data has reached the desired numerical value, the value of the operation quantity may be changed.

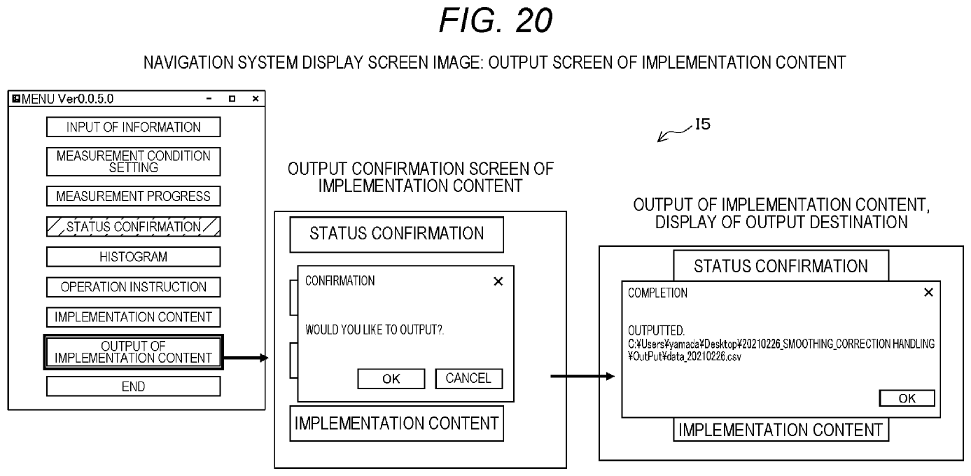

Next, the item “output of implementation content” will be described with reference to FIG. 20. FIG. 20 illustrates an example of a screen related to an output of the implementation content.

When the item of the output of the implementation content of the menu is selected, a confirmation screen I5 of necessity of output is displayed as illustrated in FIG. 20. Thereafter, when acceptance (OK) is selected, information such as the operation recommended by the navigation system and the contents performed by the worker of the sewage treatment plant can be output from the measurement result of the size and number of flocculated flocks and the water content.

When the output is completed, contents notifying that the output is completed are displayed together with the storage destination of the output file. Regarding the format of the output file, the CSV file is described in the example of the screen I5 of FIG. 20, but the file format can be appropriately selected by the system creator. Furthermore, in a case where remote control is performed, a set of output files can be transmitted to an information processing device of the remote control.

In addition to the above, as the information displayed on the screen, there is also provided a function of indicating prediction information of the flocculated flocks and the water content to the operator of the sewage treatment plant. That is, data related to future prediction may be displayed. For example, the screen I2 of “status confirmation” may display data on the predicted value of the time point at which the water content of the dried sludge is calculated or the time point after the time point at which the size or number of flocculated flocks is calculated. In this case, the operation can be performed by grasping the predicted value to be displayed.

Furthermore, data acquired by a control panel used for monitoring (monitoring) and control of equipment may be displayed on the screen. Furthermore, an image acquired by imaging the control panel may be displayed on the screen. That is, a control panel camera that images the control panel may be provided, and a monitoring value acquired by imaging the control panel and performing image processing may be displayed on the screen. By referring to the monitoring value, the information of the control panel can be easily grasped. Note that examples of the monitoring value include the amount of the chemical added to the flocculation tank, the ratio of the chemical, the concentration and flow rate of sludge, the rotation speed of the stirrer, and the pressure of the dehydrator.

Data used for numerical value display may be acquired from a sensor (for example, the sensor described above) used for monitoring or measuring each facility, or may be acquired from a control panel. Note that, in a case where the control panel displays the desired flocculation flock state, the captured image in the flocculation flock state, the water content, or the like, the displayed value or information may be obtained by the method.

The priority may be any value other than the % value as long as the facility operation contents can be appropriately evaluated. The operation quantity may be indicated by an appropriate value, and may be other than the % value.

Although the contents of the execution screen of the sewage sludge treatment facility operation assistance navigation system have been described above, words and the like to be displayed may be appropriately replaced with words and the like familiar to the operator of the sewage treatment plant. Although the sewage sludge treatment facility has been described, the system described in the embodiment may be used for sludge treatment plants other than sewage treatment, such as a flocculation tank, clean water treated using a dehydrator, and industrial wastewater.

The facility operation procedure includes facility operation contents. Then, the facility operation contents may be displayed on the display device by processing of the processor. Furthermore, the operation procedure display program may be used as an operation content display program.

### REFERENCE SIGNS LIST

100 sewage sludge treatment facility operation assistance navigation system
101 flocculation tank
102 sludge dehydrator dehydrating section
103 sludge dehydrator discharge section
104 sludge storage tank
105 imaging device
106 infrared measuring device
107 imaging signal processing device
108 infrared signal processing device
109 computer (electronic computer)
110 flocculation tank/sludge dehydrator operation procedure display device (display device)
200 sewage sludge treatment facility operation assistance navigation system
201 communication network
301 operation procedure display device screen (display screen)
401 operation procedure display device screen (display screen)
402 desired water content value (desired sludge water content)
403 water content regression line (regression line)
501 operation procedure display device screen (display screen)
502 desired water content value (desired sludge water content)
503 water content value (measurement value of water content)
601 operation procedure display device screen (display screen)
701 operation procedure display device screen (display screen)
801 operation procedure display device screen (display screen)
1100 sewage sludge treatment facility operation assistance navigation system
1101 sludge concentration tank
1102 concentrated sludge storage tank
1103 flocculation tank
1104 sludge dehydrator
1105 sludge storage tank
1106 temperature sensor
1107 sensor (temperature sensor, gas sensor)
1108 temperature sensor
1109 temperature sensor
1110 signal processing device
1111 computer (electronic computer)
1112 operation procedure display device (display device)
1200 sewage sludge treatment facility operation assistance navigation system
1201 communication network
1300 sewage sludge treatment facility operation assistance navigation system
1301 operation panel of sludge concentration tank
1302 operation panel of concentrated sludge storage tank
1303 operation panel of flocculation tank
1304 operation panel of sludge dehydrator
1305 image capturing machine
1306 image capturing machine
1307 image capturing machine
1308 image capturing machine
1309 signal processing device
1310 computer (electronic computer)
1311 operation procedure display device (display device)
1400 sewage sludge treatment facility operation assistance navigation system
1401 communication network
I1 menu screen
I2 status confirmation screen
I3 operation instruction screen
I4 implementation content input screen
I5 implementation content output screen

1. A sludge treatment facility operation assistance navigation system comprising:
a processor;
a storage resource; and
a display device,
wherein
the storage resource stores, as data,
a desired flocculation flock state that is a desired flocculation flock state in a flocculation tank of a sludge treatment facility, and
a desired sludge water content that is a desired water content of dehydrated sludge discharged from a sludge dehydrator of the sludge treatment facility, and
the processor executes an operation content display program to:
(1) acquire an operating flock state that is a flocculation flock state during operation of the sludge treatment facility;
(2) acquire an operating sludge water content that is a water content of dehydrated sludge during operation of the sludge treatment facility;
(3) select or generate facility operation contents of the flocculation tank and the sludge dehydrator based on the operating flock state, the operating sludge water content, the desired flocculation flock state, and the desired sludge water content; and
(4) display the facility operation contents on the display device.

2. The sludge treatment facility operation assistance navigation system according to claim 1, further comprising an electronic computer including the processor, the storage resource, and a communication unit that is an interface that performs communication,
wherein the electronic computer is installed in a sludge treatment plant provided with the sludge treatment facility.

3. The sludge treatment facility operation assistance navigation system according to claim 1, further comprising an electronic computer including the processor, the storage resource, and a communication unit that is an interface that performs communication,
wherein
the electronic computer is installed at a remote place different from a sludge treatment plant provided with the sludge treatment facility, and
the display device is installed in the sludge treatment plant.

4. The sludge treatment facility operation assistance navigation system according to claim 1, wherein the facility operation contents displayed on the display device include:
a procedure for adjusting a dehydration pressure of the sludge dehydrator; and
a procedure for injecting a polymer flocculant with a specified injection amount of the polymer flocculant into sewage.

5. The sludge treatment facility operation assistance navigation system according to claim 1, wherein
the storage resource stores regression line information that is data of a regression line used for predicting and calculating a water content of dehydrated sludge,
the regression line information is obtained by irradiating dehydrated sludge having a known water content with infrared rays in a wavelength range of 1200 nm or more and 2500 nm or less, performing a first differential treatment on an infrared reflectance obtained by measuring the dehydrated sludge over time, and performing a multivariate regression analysis on a result of the first differential treatment, and
the processor executes the operation content display program to
irradiate dehydrated sludge discharged from the sludge dehydrator over time with infrared rays in a wavelength range of 1200 nm or more and 2500 nm or less,
calculate the operating sludge water content using an infrared reflectance obtained by measuring the dehydrated sludge over time, and the regression line information, and
select or generate the facility operation contents based on the operating sludge water content and the desired sludge water content.

6. The sludge treatment facility operation assistance navigation system according to claim 5, wherein the desired sludge water content is expressed in a range.

7. The sludge treatment facility operation assistance navigation system according to claim 5, wherein
the dehydrated sludge having a known water content has a water content in a range of 60 wt % or more and less than 90 wt %, and
the desired sludge water content is a water content in a range of 60 wt % or more and less than 85 wt %.

8. The sludge treatment facility operation assistance navigation system according to claim 1, wherein the processor
executes a sludge volume value calculation program to calculate a sludge volume value that is a volume value of the dehydrated sludge, and display the calculated sludge volume value on the display device, and
executes a disposal cost calculation program to calculate a sludge disposal cost that is a disposal cost of the dehydrated sludge, and display the calculated sludge disposal cost on the display device.

9. The sludge treatment facility operation assistance navigation system according to claim 1, wherein the processor executes a prediction display program to predict a sludge volume value that is a volume value of the dehydrated sludge relative to an operating time of the sludge dehydrator, and predict a sludge disposal cost that is a disposal cost of the dehydrated sludge.

10. The sludge treatment facility operation assistance navigation system according to claim 1, wherein the processor executes a prediction display program to
predict a sludge volume value that is a volume value of the dehydrated sludge relative to an operating time of the sludge dehydrator and predict a sludge disposal cost that is a disposal cost of the dehydrated sludge, and
display the predicted sludge disposal cost on the display device.

11. The sludge treatment facility operation assistance navigation system according to claim 9, wherein the processor executes the prediction display program to predict the sludge disposal cost on an annual basis and display the predicted annual sludge disposal cost on the display device.

12. The sludge treatment facility operation assistance navigation system according to claim 1, wherein
the desired flocculation flock state is obtained by measuring an image of flocculated flocks flocculated in sewage over time in a predetermined area range, and performing image processing on the obtained image information to clarify a portion of the flocculated flocks and calculate a flocculated flock area that is an area of the flocculated flocks,
the desired flocculation flock state is a histogram in which a number of flocculated flocks is a frequency with the flocculated flock area as a grade,
the operating flock state is obtained by, when the sludge treatment facility is in operation, measuring an image of flocculated flocks flocculated in sewage over time in a predetermined area range, and performing the image processing on the obtained image information,
the operating flock state is a histogram in which a number of flocculated flocks is a frequency with the flocculated flock area as a grade, and
the processor executes the operation content display program to
select or generate the facility operation contents based on the operating flock state and the desired flocculation flock state, and
display the histogram of the operating flock state and the facility operation contents on the display device.

13. The sludge treatment facility operation assistance navigation system according to claim 12, wherein the flocculated flocks in the image information are an aggregate having a length range of 1 mm or more and 50 mm or less.

14. The sludge treatment facility operation assistance navigation system according to claim 1, wherein
the sludge treatment facility includes a temperature sensor that measures a temperature of the dehydrated sludge and/or a humidity sensor that measures humidity around the dehydrated sludge, and
the processor executes the operation content display program to select or generate the facility operation contents by using data acquired from the temperature sensor and/or the humidity sensor.

15. The sludge treatment facility operation assistance navigation system according to claim 1, wherein a monitoring value is obtained by capturing an image of a control panel and performing image processing.

16. The sludge treatment facility operation assistance navigation system according to claim 1, wherein the display device displays a screen for a user to input the facility operation contents after displaying the facility operation contents.

17. The sludge treatment facility operation assistance navigation system according to claim 1, wherein the processor causes the display device to display a screen including
a first area in which actual facility operation contents are input or selected by a user,
a second area in which quality of a result of implementing the facility operation contents is input or selected, and
a third area in which an implementation time is input.

18. The sludge treatment facility operation assistance navigation system according to claim 16, wherein the processor obtains a priority that is a value for recommending the facility operation contents by using information regarding the input or selected facility operation contents.

19. The sludge treatment facility operation assistance navigation system according to claim 17, wherein the processor obtains a priority that is a value for recommending the facility operation contents by using information regarding the input or selected facility operation contents.

20. The sludge treatment facility operation assistance navigation system according to claim 1, wherein
the processor obtains a predicted value of time ahead of a time point at which a water content of dehydrated sludge is calculated or a time point at which data regarding flocculated flocks is calculated, and
the display device displays the obtained predicted value.

21. The sludge treatment facility operation assistance navigation system according to claim 18, wherein the processor
receives specification of the facility operation contents on a screen displaying the facility operation contents to be implemented,
specifies an operation item and an operation quantity related to the received facility operation contents, and
inputs the specified operation item and operation quantity to the first area in which actual facility operation contents are input or selected by a user.

22. The sludge treatment facility operation assistance navigation system according to claim 19, wherein the processor
receives specification of the facility operation contents on a screen displaying the facility operation contents to be implemented,
specifies an operation item and an operation quantity related to the received facility operation contents, and
inputs the specified operation item and operation quantity to the first area.

23. The sludge treatment facility operation assistance navigation system according to claim 16, wherein the processor obtains an operation quantity of the facility operation contents to be recommended by using information regarding input or selected facility operation contents.

24. The sludge treatment facility operation assistance navigation system according to claim 17, wherein the processor obtains an operation quantity of the facility operation contents to be recommended by using information regarding input or selected facility operation contents.

25. A sludge treatment facility operation assistance method using an electronic computer, the method comprising:
(1) obtaining an operating flock state that is a flocculation flock state during operation of a sludge treatment facility;
(2) obtaining an operating sludge water content that is a water content of dehydrated sludge during operation of the sludge treatment facility;
(3) based on the operating flock state, the operating sludge water content, a desired flocculation flock state that is a desired flocculation flock state in a flocculation tank of the sludge treatment facility, and a desired sludge water content that is a desired water content of dehydrated sludge discharged from a sludge dehydrator of the sludge treatment facility, selecting or generating facility operation contents of the flocculation tank and the sludge dehydrator; and
(4) displaying the facility operation contents on a display device.

26. The sludge treatment facility operation assistance method according to claim 25, using the electronic computer at a remote place different from a sludge treatment plant provided with the sludge treatment facility, the method further comprising:
obtaining the operating flock state and the operating sludge water content via communication;
selecting or generating facility operation contents of the flocculation tank and the sludge dehydrator; and
causing the display device provided in the sludge treatment plant to display the facility operation contents via communication.

27. A program for causing an electronic computer to execute the sludge treatment facility operation assistance method according to claim 25.

28. A program for causing an electronic computer to execute the sludge treatment facility operation assistance method according to claim 26.
